SEQUENCE LISTING (10 sequences)

SEQ ID NO: 1 - protein, 24 aa, Homo sapiens
DVLAGLSSSCCKWGCSKSEISSLC

SEQ ID NO: 2 - protein, 27 aa, Homo sapiens
RAAPYGVRLCGREFIRAVIFTCGGSRW

SEQ ID NO: 3 - protein, 24 aa, Artificial Sequence
  Description of Artificial Sequence Synthetic peptide
DVLAGLSSSACKWGASKSEISSLC

SEQ ID NO: 4 - protein, 14 aa, Artificial Sequence
  Description of Artificial Sequence Synthetic peptide
CKWGASKSEISSLC

SEQ ID NO: 5 - protein, 23 aa, Artificial Sequence
  Description of Artificial Sequence Synthetic peptide
RAAPYGVRLCGREFIRAVIFTCR

SEQ ID NO: 6 - protein, 22 aa, Artificial Sequence
  Description of Artificial Sequence Synthetic peptide
GVRLCGREFIRAVIFTCGGSRW

SEQ ID NO: 7 - protein, 20 aa, Artificial Sequence
  Description of Artificial Sequence Synthetic peptide
RLCGREFIRAVIFTCGGSRW

SEQ ID NO: 8 - protein, 18 aa, Artificial Sequence
  Description of Artificial Sequence Synthetic peptide
CGREFIRAVIFTCGGSRW

SEQ ID NO: 9 - protein, 4 aa, Homo sapiens
GVRL

SEQ ID NO: 10 - protein, 5 aa, Homo sapiens
GGSRW

(19) United States
(12) Patent Application Publication
(10) Pub. No.: US 2014/0024592 A1
(43) Pub. Date: Jan. 23, 2014

(54) Modified Relaxin Polypeptides

(71) Applicants: Fazel Shabenpoor, Carlton North (AU); Mohammed Akhter Hossain, Brunswick West (AU); Ross Alexander David Bathgate, Moonee Ponds (AU); John Desmond Wade, Parkville (AU); Andrew Lawrence Gundlach, Hawthorn (AU)

(72) Inventors: Fazel Shabenpoor, Carlton North (AU); Mohammed Akhter Hossain, Brunswick West (AU); Ross Alexander David Bathgate, Moonee Ponds (AU); John Desmond Wade, Parkville (AU); Andrew Lawrence Gundlach, Hawthorn (AU)

(73) Assignee: Howard Florey Institute of Experimental Physiology and Medicine, Parkville ,Victoria (AU)

(21) Appl. No.: 13/821,747

(22) PCT Filed: Sep. 8, 2011
(86) PCT No.: PCT/AU2011/001159

§ 371 Date: Oct. 2, 2013

(30) Foreign Application Priority Data
Sep. 8, 2010 (AU) 2010904032
Jun. 17, 2011 (AU) 2011902389

(51) Int. Cl. A61K 38/22; A61K 31/7088

(52) U.S. Cl. CPC A61K 38/2221; A61K 31/7088
USPC 514/12.7; 530/399; 536/23.51; 514/44 R

(57) ABSTRACT

The present invention relates to biologically active relaxin polypeptides comprising a relaxin A chain and a B chain derived from a relaxin superfamily member, wherein the A chain comprises no intra-chain disulphide bonds. In particular embodiments the modified polypeptides comprise relaxin-3 derived A and B chains, and truncations of the A and/or B chains from the N-termini and/or C-termini. In particular embodiments the polypeptides of the invention are selective agonists or antagonists of the RXFP3 receptor.

### FIELD OF THE INVENTION

The present invention relates generally to modified relaxin polypeptides and to nucleic acids encoding the same. The present invention in particular relates to modified relaxin-3 polypeptides which selectively or specifically bind to the RXFP3 (GPCR135) receptor. The invention also relates to uses of polypeptides of the invention, methods employing the same and to compositions comprising such polypeptides.

### BACKGROUND OF THE INVENTION

Relaxin is a member of a protein hormone superfamily which also includes insulin, insulin-like grown factors I and II (IGF-I and IGF-II), and the insulin-like hormones INSL3, 4, 5 and 6. The relaxin superfamily members have a wide range of biological activities which are well described in the art.

Relaxin is a heterodimeric peptide hormone composed, in its mature form, of an A chain and a B chain linked via disulphide bridges. Human relaxins in their mature form are stabilised by three disulphide bonds, two inter-chain disulphide bonds between the A chain and B chain and one intra-chain disulphide bond between cysteine residues in the A chain.

Relaxin has been conserved through vertebrate evolution and has been characterised in a large and diverse range of vertebrate species. In particular the cysteine residues in the B and A chains responsible for the intra- and inter-chain disulphide bonds are highly conserved. Whilst in most species only two forms of relaxin have been identified (relaxin and relaxin-3), in humans three distinct forms of relaxin have been described and the genes and polypeptides characterised. These have been designated H1, H2 and H3. Homologues of H1 and H2 relaxin have been identified in other higher primates including chimpanzees, gorillas and orangutans.

Differing expression patterns for H1, H2 and H3 relaxin may suggest some differences in biological roles, however all three forms display similar biological activities, as determined for example by their ability to stimulate cAMP activity in cells expressing relaxin receptors, and accordingly share many biological functions in common.

The biological functions of relaxins include an ability to inhibit myometrial contractions, to stimulate remodelling of connective tissue and to induce softening of the tissues of the birth canal.

Additionally, relaxins increase growth and differentiation of the mammary gland and nipple and induce the breakdown of collagen, one of the main components of connective tissue. Relaxins can cause a widening of blood vessels (vasodilatation) in the kidney, mesocaecum, lung and peripheral vasculature, which leads to increased blood flow or perfusion rates in these tissues. Relaxins can also stimulate an increase in heart rate and coronary blood flow, and increase both glomerular filtration rate and renal plasma flow.

Relaxin-3 is predominantly expressed in the brain where it acts as a neuropeptide acting through its receptor RXFP3 as a regulator of homeostatic physiology and complex behaviours, including feeding and metabolism, and circadian arousal and sleep patterns, with strong interactions with brain stress and affective or mood systems.

Aberrant relaxin activity and/or expression is also implicated in a number of disorders and diseases such as, for example, cardiovascular diseases, renal diseases, fibrotic disorders (including cardiac fibrosis and fibrosis associated with airway remodelling) and neurological disorders, immune diseases and endometrial and reproductive disorders. Neurological disorders such as anxiety and depression are mental health conditions that are widespread in the community, with a lifetime prevalence estimated at 29% for anxiety disorders, and 21% for mood disorders (including depression) in the USA. Depressive disorders are currently the leading cause of ‘years-lost-to-disability’ worldwide. For many patients, current anti-anxiety and anti-depressant medications are (or become) ineffective, and even if helpful, they reduce symptoms without eliciting recovery. Therefore, more effective, targeted therapies are required. Accordingly there exist a number of important clinical applications of relaxin and relaxin agonists and antagonists.

The biological actions of relaxins are mediated through G protein coupled receptors (reviewed in Bathgate et al., 2006, Pharmacol Rev 58:7-31). To date, H1, H2 and H3 relaxins have been shown to primarily recognise and bind four receptors, RXFP1 (LGR7), RXFP2 (LGR8), RXFP3 (GPCR135) and RXFP4 (GPCR142). Interestingly, receptors RXFP1 and RXFP2 are structurally distinct from receptors RXFP3 and RXFP4, yet despite the differences there is significant cross-reactivity between different relaxin molecules and different receptors. The endogenous receptor in the brain for H3 relaxin is RXFP3, however H3 relaxin has also been shown in cell-based systems to bind and activate both RXFP1 and RXFP4. Thus, since both RXFP1 and RXFP3 are expressed in the brain, it has been very difficult to determine the precise physiological role of relaxin-3 in the brain due to its cross-activation of RXFP1.

There is a need for analogues of relaxin-3 that are selective agonists or antagonists of RXFP3, lacking the ability to bind and activate RXFP1. In view of the range of potential clinical applications of relaxin there is also a continuing need for the development of novel relaxin-3 polypeptides displaying relaxin activity, which polypeptides have improved or varied biological activity when compared to naturally occurring relaxin polypeptides and/or which display different receptor binding specificities to naturally occurring relaxin polypeptides.

### SUMMARY OF THE INVENTION

Provided herein are novel modified relaxin polypeptides having relaxin activity and/or binding activity at the relaxin receptor RXFP3, in particular which polypeptides are selective for the RXFP3 receptor over the RXFP1 receptor. Polypeptides of the invention are “modified” in that they possess A chain amino acid sequences, and optionally B chain amino acid sequences that differ from those found in corresponding native relaxin molecules at one or more positions. Typically the A chain of a modified polypeptide in accordance with an embodiment disclosed herein is derived from relaxin-3 and comprises one or more amino acid substitutions with respect the corresponding native relaxin-3 A chain such that selectivity or specificity for the RXFP3 receptor is imparted onto the mature modified relaxin polypeptide.

According to a first aspect there is provided a biologically active relaxin polypeptide comprising a relaxin A chain and a B chain derived from a relaxin superfamily member, wherein the A chain comprises no intra-chain disulphide bonds. Typically the A and B chains are linked by one or more interchain disulphide bonds. Optionally, one or more of the interchain disulphide bonds may be replaced by alternative interchain bonds, such as lactam, diselenide or dicarba bonds.

In a particular embodiment, the A chain of the polypeptide is derived from relaxin-3. Optionally the B chain is also derived from relaxin-3. The relaxin-3 may be human relaxin-3 (H3 relaxin).

The relaxin A chain may comprise one or more amino acid substitutions in which one or more cysteine residues responsible for intra-chain disulphide bond formation are replaced by different amino acids. By way of example one or both of the cysteine residues located at positions 10 and 15 of the native relaxin-3 A chain may be replaced by alanine residues. The A chain may comprise or consist of the amino acid sequence as set forth in SEQ ID NO.3, or a variant or derivative thereof. In one embodiment the polypeptide comprises an A chain comprising or consisting of the amino acid sequence set forth in SEQ ID NO:3, or a variant or derivative thereof and a B chain comprising or consisting of the amino acid sequence set forth in SEQ ID NO:2, or a variant or derivative thereof, and wherein the polypeptide is an agonist of the RXFP3 receptor.

In a particular embodiment according to the first aspect the relaxin-3 derived A chain is truncated by up to about 10 amino acids at the N-terminus compared to the native relaxin-3 A chain sequence. The A chain may comprise or consist of the amino acid sequence as set forth in SEQ ID NO.4, or a variant or derivative thereof. In one embodiment the polypeptide comprises an A chain comprising or consisting of the amino acid sequence set forth in SEQ ID NO:4, or a variant or derivative thereof and a B chain comprising or consisting of the amino acid sequence set forth in SEQ ID NO:2, or a variant or derivative thereof, and wherein the polypeptide is an agonist of the RXFP3 receptor.

The relaxin-3 derived B chain may also be truncated by one or more residues at the N-terminus compared to the native relaxin-3 B chain sequence. In an embodiment, the B chain is truncated by up to about 5 residues at the N-terminus, up to about 7 residues at the N-terminus, or up to about 9 residues at the N-terminus. In a particular embodiment the polypeptide comprises an A chain comprising or consisting of the amino acid sequence set forth in SEQ ID NO:4, or a variant or derivative thereof and a B chain comprising or consisting of the amino acid sequence set forth in SEQ ID NO:6, SEQ ID NO:7 or SEQ ID NO:8, or a variant or derivative thereof, and wherein the polypeptide is an agonist of the RXFP3 receptor.

In a further exemplary modified polypeptide according to the first aspect: (i) the relaxin-3 derived A chain is truncated by up to about 10 amino acids at the N-terminus compared to the native relaxin-3 A chain sequence; (ii) the relaxin-3 derived B chain is truncated by about 5 amino acids at the C-terminus compared to the native relaxin-3 B chain sequence; and optionally (iii) a basic amino acid is incorporated at the C-terminus of the relaxin-3 derived B chain. The basic amino acid may be arginine. In one embodiment, the C-terminal 5 amino acids from the native sequence of the relaxin-3 B chain are replaced by a terminal arginine residue. The B chain may comprise or consist of the amino acid sequence set forth in SEQ ID NO:5, or a variant or derivative thereof. In one embodiment the polypeptide comprises an A chain comprising or consisting of the amino acid sequence set forth in SEQ ID NO:4, or a variant or derivative thereof and a B chain comprising or consisting of the amino acid sequence set forth in SEQ ID NO:5, or a variant or derivative thereof, and wherein the polypeptide is an antagonist of the RXFP3 receptor.

In particular embodiments, relaxin polypeptides of the first aspect selectively bind to the RXFP3 receptor. In further particular embodiments relaxin polypeptides of the first aspect may be specific for the RXFP3 receptor.

According to a second aspect there is provided a biologically active relaxin polypeptide selective for the RXFP3 receptor, wherein the polypeptide comprises an A chain derived from relaxin-3 and a B chain derived from a relaxin superfamily member, wherein the A chain comprises no intra-chain disulphide bonds and is truncated by up to 10 amino acids at the N-terminus when compared to the corresponding native relaxin-3 A chain sequence.

In a particular embodiment, the B chain is derived from relaxin-3. The relaxin-3 may be human relaxin-3 (H3 relaxin). In an embodiment the B chain is truncated by up to 5 residues at the N-terminus when compared to the corresponding native relaxin-3 B chain sequence. In other embodiments the B chain is truncated by up to 7 residues or up to 9 residues at the N-terminus when compared to the corresponding native relaxin-3 B chain sequence.

In an embodiment, the relaxin polypeptide according to the second aspect is an agonist of the RXFP3 receptor.

According to a third aspect there is provided a biologically active relaxin polypeptide comprising a relaxin A chain and a B chain derived from a relaxin superfamily member, wherein the A chain comprises no intra-chain disulphide bonds and wherein the B chain comprises a truncation of one or more amino acids from the C-terminus compared to the corresponding native sequence. Optionally, the A chain is derived from relaxin-3 and is truncated by up to 10 amino acids at the N-terminus when compared to the corresponding native relaxin-3 A chain sequence.

In a particular embodiment, the B chain is derived from relaxin-3. The relaxin-3 may be human relaxin-3 (H3 relaxin).

The B chain may be truncated by, for example, five amino acid residues at the C-terminus. In an embodiment the terminal five amino acid residues of the B chain derived from relaxin-3 are deleted and replaced by a basic amino acid residues, for example an arginine residue.

In an embodiment, the relaxin polypeptide according to the third aspect is an antagonist of the RXFP3 receptor.

A fourth aspect provides polynucleotides encoding modified relaxin polypeptides according to the first, second and third aspects.

A fifth aspect provides a pharmaceutical composition comprising a modified relaxin polypeptide of the first, second or third aspect or a polynucleotide of the fourth aspect, optionally together with one or more pharmaceutically acceptable carriers, excipients or diluents.

A sixth aspect provides a method for treating or preventing a disease or condition, the method comprising administering to a subject in need thereof a modified relaxin polypeptide of the first, second or third aspect, a polynucleotide of the fourth aspect or a pharmaceutical composition of the fifth aspect.

In an embodiment the disease or condition may be associated with aberrant expression and/or activity of H3 relaxin or with aberrant expression and/or activity of the RXFP3 receptor. In an alternative embodiment, a modified polypeptide of the invention (or a polynucleotide encoding the same, or a pharmaceutical composition comprising the same) may be administered for the treatment or prevention of a disease or condition wherein the inhibition of biological activity at, or signalling via, the RXFP3 receptor is desirable.

In exemplary embodiments the polypeptide is an agonist of the RXFP3 receptor in accordance with the second aspect and the disease or condition is selected from, or is associated with, vascular disease including coronary artery disease, peripheral vascular disease, vasospasm including Raynaud's phenomenon, microvascular disease involving the central and peripheral nervous system, kidney, eye and other organs; treatment of arterial hypertension; diseases related to uncontrolled or abnormal collagen or fibronectin formation such as fibrotic disorders (including fibrosis of lung, heart and cardiovascular system, kidney and genitourinary tract, gastrointestinal system, cutaneous, rheumatologic and hepatobiliary systems); kidney disease associated with vascular disease, interstitial fibrosis, glomerulosclerosis, or other kidney disorders; psychiatric disorders including circadian/sleep disorders in adolescents, adults and the aged, including insomnia; stress, anxiety disorders including global anxiety, chronic anxiety, acute stress disorder, agoraphobia (with or without a history of panic disorder), generalized anxiety disorder (GAD), obsessive-compulsive disorder (OCD), panic disorder (with or without agoraphobia), phobias (including social phobia) and posttraumatic stress disorder (PTSD); mood disorders including bipolar disorder, unipolar disorder, cyclothymic disorder, dysthymic disorder, depression, major depressive disorder; depressive symptoms of the neurodegenerative diseases; neurologic or neurodegenerative diseases (including memory loss or other memory disorders, dementias, Alzheimer's disease); neurodevelopmental disorders of Autism and Autism Spectrum Disorders (ASD), disorders of learning, attention and motivation (including Attention Deficit Hyperactivity Disorder, Tourette's disease, impulsivity, obsessive compulsive disorders, antisocial and personality disorders, negative symptoms of psychoses including those due to schizophrenia, acquired brain damage and frontal lobe lesions); addictive disorders (including drug, alcohol and nicotine addiction); movement and locomotor disorders (including Parkinson's disease, Huntington's disease and their depressive and cognitive symptoms, and motor deficits after stoke, head injury, surgery, tumour or spinal cord injury); immunological disorders (including immune deficiency states, haematological and reticuloendothelial malignancy; breast disorders (including fibrocystic disease, impaired lactation, and cancer); endometrial disorders including infertility due to impaired implantation; endocrine disorders (including adrenal, ovarian and testicular disorders related to steroid or peptide hormone production); delayed onset of labour, impaired cervical ripening, and prevention of prolonged labour due to fetal dystocia; sinus bradycardia; hair loss, alopecia; disorders of water balance including impaired or inappropriate secretion of vasopressin; and placental insufficiency.

In further exemplary embodiments the polypeptide is an antagonist of the RXFP3 receptor in accordance with the third aspect and the disease or condition is selected from, or is associated with, substance use, abuse and/or addiction, addictive behaviour and symptoms and conditions associated with substance abuse and addiction, Attention Deficit Hyperactivity Disorder (ADHD), obsessive compulsory disorder, developmental disorders such as autism and Autism Spectrum Disorders (ASD), neuroendocrine disorders of pregnancy and post-partum period (postnatal depression), medication-related hyperactivity or hyper-arousal conditions, stress, anxiety disorders, mood disorders, and depressive symptoms of the neurodegenerative diseases.

The anxiety disorders may be selected from the group comprising; global anxiety, chronic anxiety, acute stress disorder, agoraphobia (with or without a history of panic disorder), generalized anxiety disorder (GAD), obsessive-compulsive disorder (OCD), panic disorder (with or without agoraphobia), phobias (including social phobia) and posttraumatic stress disorder (PTSD).

The mood disorder may be selected from the group comprising; bipolar disorder, unipolar disorder, cyclothymic disorder, dysthymic disorder, depression, and major depressive disorder.

A seventh aspect provides a method for treating or preventing anxiety-related behaviours, the method comprising administering to a subject in need thereof a modified relaxin polypeptide of the first, second or third aspect, a polynucleotide of the fourth aspect or a pharmaceutical composition of the fifth aspect.

An eighth aspect provides the use of a modified relaxin polypeptide of the first, second or third aspect, or a polynucleotide of the fourth aspect for the manufacture of a medicament for the treatment or prevention of a disease or condition, optionally a condition or disorder associated with aberrant expression and/or activity of H3 relaxin or with aberrant expression and/or activity of the RXFP3 receptor, or wherein the inhibition of biological activity at, or signalling via, the RXFP3 receptor is desirable.

Also provided is the use of a modified relaxin polypeptide of the first, second or third aspect, or a polynucleotide of the fourth aspect in a method for the treatment or prevention of a disease or condition, optionally a condition or disorder associated with aberrant expression and/or activity of H3 relaxin or with aberrant expression and/or activity of the RXFP3 receptor, or wherein the inhibition of biological activity at, or signalling via, the RXFP3 receptor is desirable.

### BRIEF DESCRIPTION OF THE DRAWINGS

The present invention is described, by way of non-limiting example only, with reference to the accompanying drawings.

FIG. 1. Schematic illustration of the generation of exemplary modified relaxin-3 polypeptides (herein designated analogue 14, analogue 15 and analogue 17) according to embodiments of the invention. Modelled three-dimensional structures and primary amino acid sequences of native human relaxin-3 (H3) and analogues 14, 15 and 17 are shown. Each polypeptide is dimeric, with the sequence of the A chain shown above the sequence of the B chain. Intra- and inter-chain disulphide bonds between cysteine residues are shown as solid black lines. Amino acid residues in bold type represent amino acids altered with respect to the native H3 relaxin sequence. Analogues 18 and 19 (not shown) were generated by truncating the B chain present in analogue 17 by 2 residues (ΔGV) and 4 residues (ΔGVRL), respectively, from the N-terminus.

FIG. 2. cAMP activity as a measure of activation of RXFP3 receptor by relaxin-2, relaxin-3 and exemplary modified relaxin-3 polypeptides (analogues 14, 15 and 17).

FIG. 3. cAMP activity as a measure of activation of RXFP1 receptor by relaxin-2, relaxin-3 and exemplary modified relaxin-3 polypeptides (analogues 14 and 15).

FIG. 4. Effect of H2 relaxin (H2), H3 relaxin (H3) and analogue 15 (15) on TGF-β1 (T)-induced collagen accumulation in human dermo-fibroblast cells expressing RXFP1. H2 relaxin and H3 relaxin inhibited the expression of TFG-β1 induced collagen by 35% and 38%, respectively compared to TGF-β1 alone. In contrast, analogue 15, did not alter collagen expression (n=3-4 per group, ** P<0.01 vs control, # P<0.05 vs TGF-β1).

FIG. 5. Primary sequence of relaxin-3, relationship between the sequences of exemplary modified relaxin-3 polypeptides, and generation of exemplary modified relaxin-3 polypeptide analogue 16. Deletion of the intra-A-chain disulfide bond leads to analogue 14 and further truncation of 10 residues from the N-terminus of the A-chain resulted in analogue 15. Analogue 15 was in turn was modified at the C-terminus of its B-chain by replacing GGSRW residues with R to give analogue 16, and at the N-terminus of its B chain by deletion of 5 amino acid residues to give analogue 17.

FIG. 6. Analogues 15 and 16 bind to RXFP3 with similar affinity as native relaxin-3 (A). Unlike analogue 15, a selective agonist of RXFP3, analogue 16 is an antagonist (B) of this receptor. Relaxin-3 at 10 nM inhibited forskolin-induced cAMP production in CHO-K1 cells. Addition of increasing concentrations of analogue 16 rescued the level of cAMP back to about 100% by binding to RXFP3 and preventing its activation by relaxin-3.

FIG. 7. Effect of central administration of analogues 15 and 16 on food intake in satiated, adult male Sprague-Dawley rats. Effect of intracerebroventricular administration of vehicle (aCSF, 5 μl) or mock injection (V/Con), R3/15 (˜1 nmol; 5 μg in 5 μl aCSF), analogue 15 (˜1.1 nmol; 5 μg in 5 μl aCSF; A2), analogue 16 (˜4.8 nmol; 20 μg in 2.5 μl aCSF; A3), and analogue 16 (10 min prior)+analogue 15 or aCSF vehicle on food intake, during the first 60 min post-injection. Feeding experiments were performed in different groups of rats over several days with a crossover design, and the results were combined (n=7-11 per group; ** P<0.01 vs control, *** P<0.001 vs 15).

FIG. 8. Effect of acute intracerebroventricular administration of 5 μg of analogue 15, (designated RXFP3-A2 in this Figure), versus aCSF (control) on behaviour of adult male Sprague-Dawley rats in a light-dark box. (A) Entries into the light compartment. (B) Time spent in the light compartment. (C) Number of moves in the light compartment. (D) Latency to enter the light compartment. (Note: Latency scores exclude rats that do not enter the light side). Data represent mean±SEM. Statistical significance evaluated using a Student's t-test—* P<0.05; ** P<0.01; *** P<0.001. Numbers in columns indicate group size.

FIG. 9. Effect of acute intracerebroventricular administration of 5 μg of analogue 15, (designated RXFP3-A2 in this Figure), versus aCSF (control) on behaviour of adult, male Sprague-Dawley rats in the elevated plus maze. (A) Entries into the open arms (as a percentage of the total number of arm entries). (B) Time spent in the open arms (as a percentage of the total time on both open and closed arms). (C) Total number of entries into the closed arms. Data represent mean±SEM. Statistical significance evaluated using a Student's t-test—* P<0.05; ** P<0.01; *** P<0.001; ns, not significant. Numbers in columns indicate group size.

FIG. 10. Comparison of the effect of history (pre-testing vs naïve) and treatment (control vs analogue 15, designated RXFP3-A2 in this Figure) on the immobility time of adult, male Sprague-Dawley rats in the forced swim test. Data represent mean±SEM. Statistical significance was evaluated using a two-way ANOVA, with a Bonferroni post-hoc test—** P<0.01. Numbers in columns indicate group size.

FIG. 11. Effect of previous anxiety testing (‘pre-tested’) versus no previous testing (‘naïve’) on the behaviour of adult, male Sprague-Dawley rats in the forced swim test. Data represent mean±SEM of the total duration spent immobile in the ‘Porsolt posture’. Statistical significance was evaluated using a Student's t-test—* P<0.05. Numbers in columns indicate group size.

Amino acid sequences of native human relaxin-3 A and B chains are set forth in SEQ ID NOs: 1 and 2, respectively. SEQ ID NO: 3 provides the amino acid sequence of the A chain of modified polypeptide analogue 14 described and exemplified herein. SEQ ID NO: 4 provides the amino acid sequence of the A chain of modified polypeptide analogue 15 described and exemplified herein. SEQ ID NO: 5 provides the amino acid sequence of the B chain of modified polypeptide analogue 16 described and exemplified herein. SEQ ID NO: 6 provides the amino acid sequence of the B chain of modified polypeptide analogue 17 described and exemplified herein. SEQ ID NO: 7 provides the amino acid sequence of the B chain of modified polypeptide analogue 18 described and exemplified herein. SEQ ID NO: 8 provides the amino acid sequence of the B chain of modified polypeptide analogue 19 described and exemplified herein. Thus, the A and B chain amino acid sequences of the exemplary modified polypeptide analogues 14-19 described herein are as follows:

Analogue 14—A chain (SEQ ID NO:3)+B chain (SEQ ID NO:2)
Analogue 15—A chain (SEQ ID NO:4)+B chain (SEQ ID NO:2)
Analogue 16—A chain (SEQ ID NO:4)+B chain (SEQ ID NO:5)
Analogue 17—A chain (SEQ ID NO:4)+B chain (SEQ ID NO:6)
Analogue 18—A chain (SEQ ID NO:4)+B chain (SEQ ID NO:7)
Analogue 19—A chain (SEQ ID NO:4)+B chain (SEQ ID NO:8)

### DETAILED DESCRIPTION OF THE INVENTION

The articles “a” and “an” are used herein to refer to one or to more than one (i.e., to at least one) of the grammatical object of the article. By way of example, “an element” means one element or more than one element.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

In the context of this specification, the term “about,” is understood to refer to a range of numbers that a person of skill in the art would consider equivalent to the recited value in the context of achieving the same function or result.

The term “polypeptide” means a polymer made up of amino acids linked together by peptide bonds. The term “peptide” may also be used to refer to such a polymer although in some instances a peptide may be shorter (i.e. composed of fewer amino acid residues) than a polypeptide. Nevertheless, the terms “polypeptide” and “peptide” are used interchangeably herein.

The term “relaxin polypeptide” as used herein means a polypeptide, whether modified in accordance with the present invention or corresponding to a naturally occurring relaxin molecule which displays biological activity typically associated with relaxin. The level of such relaxin biological activity displayed by a modified polypeptide of the invention may be equivalent to that of a naturally occurring or native relaxin, or may be enhanced or reduced when compared with the activity of a naturally occurring or native relaxin. The “biological activity” typically comprises the ability to bind a relaxin receptor such as for example RXFP3; it is not necessary for the polypeptide to induce a response of the same type as that induced by a naturally occurring relaxin bound to the same receptor. A relaxin polypeptide in accordance with the present disclosure may either agonise or antagonise the receptor. Thus for a polypeptide to be regarded as a “biologically active” relaxin polypeptide it is not necessary that the nature of the biological or physiological response induced by the polypeptide via a receptor be the same as that of the corresponding naturally occurring relaxin molecule. In the context of the present disclosure, the term relaxin polypeptide refers to heterodimeric polypeptides comprising an A chain and a B chain.

The term “modified” as used herein in the context of a relaxin polypeptide means a polypeptide that differs from a naturally occurring or native relaxin polypeptide at one or more amino acid positions in one or more peptide chains of such naturally occurring or native polypeptide.

The term “conservative amino acid substitution” as used herein refers to a substitution or replacement of one amino acid for another amino acid with similar properties within a polypeptide chain (primary sequence of a protein). For example, the substitution of the charged amino acid glutamic acid (Glu) for the similarly charged amino acid aspartic acid (Asp) would be a conservative amino acid substitution. The nature of other conservative amino acid substitutions are well known to those skilled in the art.

As used herein the term “derived” in the context of relaxin A and B chains in modified polypeptides means that the A and B chain sequences correspond to, originate from, or otherwise share significant sequence homology with naturally occurring A and B chain sequences. Thus, for example, a relaxin B chain present in a modified polypeptide may be identical to the B chain sequence of a relaxin or relaxin superfamily member from any species or may be a modified version or variant thereof. Alternatively, the A chain in a modified polypeptide may share sequence homology with one or more A chain sequences from any species. In the context of relaxin polypeptides the terms “naturally occurring” and “native” refer to relaxin polypeptides as encoded by and produced from the genome of an organism. For example in humans three distinct forms of relaxin have been identified to date, H1, H2 and H3. Each of these forms is considered herein as a different “naturally occurring” or “native” relaxin. Those skilled in the art will also understand that by being “derived” from a naturally occurring or native relaxin sequence, the sequence in the modified polypeptide need not be physically constructed or generated from the naturally occurring or native sequence, but may be chemically synthesised such that the sequence is “derived” from the naturally occurring or native sequence in that it shares sequence homology and function with the naturally occurring or native sequence.

As used herein the term “selective” when used in the context of the ability of a modified relaxin polypeptide to bind the RXFP3 receptor, or the ability of a polypeptide to act as an agonist or antagonist of RXFP3 receptor function, means that the polypeptide binds the RXFP3 receptor at significantly higher frequency than it binds other receptors, in particular the RXFP1 receptor, or the polypeptide agonises or antagonises RXFP3 to a significantly greater extent than it agonises or antagonises other receptors, in particular the RXFP1 receptor. A modified relaxin polypeptide that is “specific” for the RXFP3 receptor is one that possesses no discernable activity at any other receptor. Thus, a modified relaxin polypeptide that is “specific” for RXFP3 is, by definition, selective for RXFP3.

The term “polynucleotide” as used herein refers to a single- or double-stranded polymer of deoxyribonucleotide, ribonucleotide bases or known analogues of natural nucleotides, or mixtures thereof. The term includes reference to the specified sequence as well as to the sequence complimentary thereto, unless otherwise indicated. The terms “polynucleotide” and “nucleic acid” are used interchangeably herein.

As used herein the terms “treating”, “treatment”, “preventing” and “prevention” refer to any and all uses which remedy a disease, disorder or condition or symptoms, prevent the establishment of a condition or disease, or otherwise prevent, hinder, retard, or reverse the progression of a condition or disease or other undesirable symptoms in any way whatsoever. Thus the terms “treating” and “preventing” and the like are to be considered in their broadest context. For example, treatment does not necessarily imply that a patient is treated until total recovery. Similarly, “prevention” does not necessarily mean that the subject will not eventually contract a particular disease, disorder or condition. Rather, “prevention” encompasses reducing the severity of, or delaying the onset of, a particular disease, disorder or condition. In the context of some conditions, methods of the present invention involve “treating” the disease, disorder or condition in terms of reducing or eliminating the occurrence of a highly undesirable and irreversible outcome of the progression of the condition but may not of itself prevent the initial occurrence of the disease, disorder or condition. Accordingly, treatment and prevention include amelioration of the symptoms of a particular disease, disorder or condition or preventing or otherwise reducing the risk of developing a particular disease, disorder or condition.

As used herein the term “anxiety-related behaviour” refers to any behaviour associated with, causative of or resulting from a feeling of “anxiety” in an individual. In particular, an anxiety-related behaviour is any behaviour associated with an aversive stimulus in a normal subject. Anxiety is a normal behaviour. For example, a loud noise generates a ‘fright’ response with changes in body physiology such as increased heart rate, tensed muscles, and an acute sense of focus in an effort to determine the source of the noise. The latter changes are part of the natural ‘flight or flight’ phenomenon whereby the body prepares itself to either fight or protect itself, or to flee a dangerous situation; and these changes are all symptoms of anxiety. Such symptoms become a clinical problem or an “anxiety disorder” when they occur without any recognizable stimulus or when the stimulus does not warrant such a reaction (i.e. inappropriate anxiety is when a person's heart races, breathing increases, and muscles tense without any reason for them to do so). Human anxiety disorders are typically characterized by a primary feature of abnormal or inappropriate anxiety.

As used herein the term “depressive-related behaviour” refers to any behaviour associated with, causative of or resulting from a feeling of “depression” in an individual, such as mood disorders. Mood disorders are those where the primary symptom is a disturbance in mood or “an inappropriate, exaggerated, or limited range of feelings”. To be diagnosed as suffering a mood disorder, feelings must be “extreme” (i.e. crying, and/or feeling depressed, suicidal frequently; or the opposite extreme, excessive energy where sleep is not needed for days at a time and during this time the decision making process in significantly hindered).

As used herein the terms “effective amount” and “effective dose” include within their meaning a non-toxic but sufficient amount or dose of an agent or compound to provide the desired effect. The exact amount or dose required will vary from subject to subject depending on factors such as the species being treated, the age and general condition of the subject, the severity of the disease, disorder or condition being treated, the particular agent being administered and the mode of administration and so forth. Thus, it is not possible to specify an exact “effective amount” or “effective dose”. However, for any given case, an appropriate “effective amount” or “effective dose” may be determined by one of ordinary skill in the art using only routine experimentation.

The development of potent and selective RXFP3 agonists and antagonists, that are synthetically tractable, to facilitate the in vivo characterisation of the neurological role of relaxin-3/RXFP3 and to realise the potential of RXFP3 as a pharmaceutical target, has been an important challenge for relaxin researchers.

During the course of ongoing structure-function relationship studies of human relaxin-3, the present inventors observed that significant chain truncation and subsequent minimization of the relaxin-3 polypeptide could be achieved without undue loss of RXFP3 binding and activation. Remarkably, this propensity was also retained following removal of the intramolecular disulfide bond of the A-chain. Moreover, as exemplified herein, by such manipulation of the relaxin-3 A chain the inventors have successfully developed for the first time modified relaxin-3 polypeptides displaying strong selectivity for RXFP3.

Provided herein are biologically active relaxin polypeptides comprising a relaxin A chain and a B chain derived from a relaxin superfamily member, wherein the A chain comprises no intra-chain disulphide bonds. Typically the modified polypeptide comprises an A chain and a B chain derived from relaxin-3. Also typically the modified polypeptide is selective or specific for the RXFP3 receptor.

Also provided herein are biologically active relaxin polypeptides selective for the RXFP3 receptor, wherein the polypeptide comprises an A chain derived from relaxin-3 and a B chain derived from a relaxin superfamily member, wherein the A chain comprises no intra-chain disulphide bonds and is truncated by up to 10 amino acids at the N-terminus when compared to the corresponding native relaxin-3 A chain sequence. Typically the modified polypeptide comprises an A chain and a B chain derived from relaxin-3. The modified polypeptide may further comprise a truncation of up to about 5 amino acids from the N-terminus of the B chain or from the C-terminus of the B chain. Where the truncation is at the C-terminus of the B chain the deleted amino acid residues may be replaced with a basic residue such as an arginine. The modified polypeptide may be specific for the RXFP3 receptor and may be an agonist or an antagonist of the RXFP3 receptor.

An advantage of certain modified relaxin-3 polypeptides exemplified herein is that they are considerably simpler in structure than the native human relaxin-3, being smaller and possessing one less disulfide bond, and yet retain biological activity. Modified relaxin-3 polypeptides of the present disclosure that are agonists of the RXFP3 receptor possess virtually equipotent activity as the native human relaxin-3. The exemplified modified polypeptides are the most selective relaxin-3 molecules described to date.

Described and exemplified herein are modified polypeptides in which the N-terminus of the A chain is truncated by up to about 10 amino acid residues. In a specific embodiment the N-terminus of the A chain is truncated by 10 amino acids, however those skilled in the art will appreciate that the scope of the present disclosure is not so limited and less than 10 amino acids may be removed without departing from the scope of the disclosure. By way of example only the truncation at the N-terminus may be of one or more amino acids, typically of 2, 3, 4, 5, 6, 7, 8, 9 or 10 amino acids. Similarly, where the A chain of the modified polypeptide is derived from H3 relaxin, typically no more than 10 amino acids are removed from the N-terminus as the cysteine residue at position 11 of the H3 A chain amino acid sequence (SEQ ID NO.1) is required for inter-chain disulphide bond formation. However in other embodiments, where the cysteine residue responsible for inter-chain disulphide bond formation is located further from the N-terminus than in H3 relaxin, more than 10 amino acids may be removed from the N-terminus of the A chain in generating modified polypeptides in accordance with the present disclosure.

Also described and exemplified herein are modified polypeptides in which the N-terminus and/or the C-terminus of the B chain is truncated by up to about 9 amino acid residues. However those skilled in the art will appreciate that the scope of the present disclosure is not so limited and more or less than 9 amino acids may be removed without departing from the scope of the disclosure. By way of example only, the truncation at the N-terminus or C-terminus of the B chain may be of one or more amino acids, typically of 2, 3, 4, 5, 6, 7, 8 or 9 amino acids.

In the modified polypeptides of the present disclosure the A chain amino acid sequence is typically derived from a relaxin-3 molecule, more typically from human relaxin-3 (H3 relaxin). The B chain may be derived from any relaxin superfamily member. In a particular embodiment, both the A and the B chain are derived from relaxin-3 molecules, typically H3 relaxin. The A chain of native H3 relaxin comprises the amino acid sequence depicted in SEQ ID NO.1 and the B chain of native H3 relaxin comprises the amino acid sequence depicted in SEQ ID NO.2. Accordingly, the A and B chain amino acid sequences of modified relaxin polypeptides the subject of the present disclosure may be based on or derived from the amino acid sequences of the A and B chains of H3 relaxin, for example those depicted in SEQ ID NO:1 and SEQ ID NO:2, respectively. However those skilled in the art will also appreciate that the amino acid sequences of the A and/or B chains from which the modified polypeptides of the invention may be based, or from which the modified polypeptides may be derived, may include variants of these relaxin-3 sequences.

The term “variant” as used herein refers to substantially similar sequences. Generally, polypeptide sequence variants also possess qualitative biological activity in common, such as receptor binding activity. Further, these polypeptide sequence variants may share at least 50%, 55%, 60%, 65%, 70%, 75%, 80%, 85%, 90%, 91%, 92%, 93%, 94%, 95%, 96%, 97%, 98% or 99% sequence identity. Also included within the meaning of the term “variant” are homologues of polypeptides of the disclosure. A homologue is typically a polypeptide from a different species but sharing substantially the same biological function or activity as the corresponding polypeptide disclosed herein. Further, the term “variant” also includes analogues of the polypeptides of the present disclosure, wherein the term “analogue” means a polypeptide which is a derivative of a polypeptide of the disclosure, which derivative comprises addition, deletion, substitution of one or more amino acids, such that the polypeptide typically retains substantially the same function, for example in terms of receptor binding activity. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in a polypeptide although random insertion is also possible with suitable screening of the resulting product. Deletional variants are characterised by the removal of one or more amino acids from the sequence. Substitutional amino acid variants are those in which at least one residue in a sequence has been removed and a different residue inserted in its place. Additions to amino acid sequences may include fusions with other peptides, polypeptides or proteins. Modifications may be made to relaxin polynucleotide sequences, for example via the insertion or deletion of one or more codons, such that modified derivatives of the relaxin polypeptide are generated. Such modifications are also included within the scope of the term “variant”. For example, modifications may be made so as to enhance the biological activity or expression level of the relaxin or to otherwise increase the effectiveness of the polypeptide to achieve a desired outcome.

In particular exemplary embodiments the A chain of a modified relaxin-3 polypeptide of the present disclosure comprises or consists of the amino acid sequence depicted in SEQ ID NO.3 or SEQ ID N0.4, or a variant or derivative thereof.

As exemplified herein it is modifications made to the A chain of relaxin-3 that impart RXFP3 selectivity or specificity onto the modified relaxin-3 polypeptides. Where the B chain of the modified polypeptide is retained intact and unchanged when compared to the native sequence, the modified polypeptide selectively or specifically binds and activates biological activity via RXFP3 (a selective or specific agonist of RXFP3).

However also provided are modified relaxin polypeptides capable of selectively or specifically binding RXFP3 and inhibiting the function of this receptor (selective or specific antagonists of RXFP3). Thus, in another aspect of the present disclosure there is provided a biologically active relaxin polypeptide comprising a relaxin A chain and a B chain derived from a relaxin superfamily member, wherein the A chain comprises no intra-chain disulphide bonds and wherein the B chain comprises a truncation of one or more amino acids from the C-terminus compared to the corresponding native sequence. The B chain may be truncated by, for example, five amino acid residues at the C-terminus. Further, the residues deleted from the C-terminus may be replaced by a basic amino acid residue. The basic amino acid may be arginine. In an embodiment the terminal five amino acid residues of the B chain derived from relaxin-3 are deleted and replaced by a single arginine residue. Thus, in an exemplary embodiment the B chain of a modified polypeptide according the present disclosure comprises or consists of the amino acid sequence as set forth in SEQ ID NO:5, or a variant or derivative thereof. The A chain of such an antagonist polypeptide may also be truncated with respect to the corresponding naturally occurring relaxin A chain, typically by up to about 10 amino acids. Thus, an antagonist polypeptide exemplified herein comprises a B chain amino acid sequence as set forth in SEQ ID NO:5, or a variant or derivative thereof and an A chain amino acid sequence as set forth in SEQ ID NO:4, or a variant or derivative thereof.

In the modified polypeptides of the present disclosure the A chain, and optionally the B chain may be modified from those found in a naturally occurring or native relaxin molecules by any number of means well known to those skilled in the art. For example the amino acid sequences may be modified by one or more amino acid insertions, deletions and/or substitutions using recombinant DNA and molecular biology techniques known to those skilled in the art. Further, whilst the A and B chains of modified polypeptides of the present disclosure are typically linked by one or more interchain disulphide bonds, alternative interchain linkages are also contemplated. By way of example, one or more of the interchain disulphide bonds may be replaced by an alternative bond such as a lactam, diselenide or dicarba bond to increase plasma stability of the modified polypeptide. Those skilled in the art will appreciate that a range of other bonds or linkages may be employed in place of one or more of the interchain disulphide bonds (and that the present disclosure is not limited by reference to the exemplary bonds/linkages specifically described herein), and that a number of alternative modifications may be made to increase the plasma stability of the modified polypeptides.

The present disclosure contemplates modified relaxin polypeptides in which the A and/or B chains possess one or more amino acid deletions, additions or substitutions in comparison with a corresponding native relaxin polypeptide. Amino acid changes in relaxin polypeptides may be effected by techniques well known to those persons skilled in the relevant art. For example, amino acid changes may be effected by nucleotide replacement techniques which include the addition, deletion or substitution of nucleotides (conservative and/or non-conservative), under the proviso that the proper reading frame is maintained. A conservative substitution denotes the replacement of an amino acid residue by another, biologically similar residue. Examples of conservative substitutions include the substitution of one hydrophobic residue such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic acid for aspartic acid, or glutamine for asparagine, and the like. Neutral hydrophilic amino acids which can be substituted for one another include asparagine, glutamine, serine and threonine. The term “conservative substitution” also includes the use of a substituted amino acid in place of an unsubstituted parent amino acid. Exemplary techniques for generating such amino acid insertion, deletion or substitution modifications include random mutagenesis, site-directed mutagenesis, oligonucleotide-mediated or polynucleotide-mediated mutagenesis, deletion of selected region(s) through the use of existing or engineered restriction enzyme sites, and the polymerase chain reaction. Such techniques will be well known to those skilled in the art.

Polypeptides of the disclosure can also be further modified, for instance, by glycosylation, amidation, carboxylation, or phosphorylation, or by the creation of acid addition salts, amides, esters, in particular C-terminal esters, and N-acyl derivatives of the polypeptides. The polypeptides can also be further modified to create polypeptide derivatives by forming covalent or noncovalent complexes with other moieties. Covalently-bound complexes can be prepared by cross-linking the chemical moieties to functional groups on the side chains of amino acids comprising the peptides, or at the N— or C terminus. For example, as polypeptide sequence minimisation is often accompanied by increased susceptibility to enzymatic attack and degradation with a corresponding decrease in plasma half life and in vivo activity, a modified polypeptide of the present disclosure may be generated with a polyethylene moiety conjugated at one or more locations (PEGylation) to increase in vivo half life of the polypeptide. Those skilled in the art will appreciate that a number of other well known approaches exist to extend the in vivo half life of polypeptides, such as for example the addition of albumin affinity tags, and the present disclosure is not limited by reference to the exemplary means specifically discussed herein.

Further, the polypeptides of the present disclosure can be conjugated to a reporter group, including, but not limited to a radiolabel, a fluorescent label, an enzyme (e.g., that catalyzes a colorimetric or fluorometric reaction), a substrate, a solid matrix, or a carrier (e.g., biotin or avidin). These are merely exemplary additional modifications that may be made to the modified polypeptides of the invention. Those skilled in the art will appreciate that further modifications may also be made so as to generate analogues of the polypeptides of the present disclosure. By way of example only, illustrative analogues and processes for preparing the same are described in International patent application published as WO 2004/113381, the disclosure of which is incorporated herein by reference in its entirety.

Amino acid additions may also result from the fusion of a relaxin polypeptide or fragment thereof with a second polypeptide or peptide, such as a polyhistidine tag, maltose binding protein fusion, glutathione S transferase fusion, green fluorescent protein fusion, or the addition of an epitope tag such as FLAG or c-myc. Additionally, modified polypeptides may further include a relaxin C chain. Relaxin C chain amino acid sequences are known to those skilled in the art.

The present disclosure also contemplates fragments and variants of the polypeptides disclosed herein.

The term “fragment” refers to a polypeptide molecule that is a constituent of a polypeptide of the disclosure or variant thereof. Typically the fragment possesses qualitative biological activity in common with the polypeptide of which it is a constituent. The peptide fragment may be between about 5 to about 26 amino acids in length, between about 5 to about 25 amino acids in length, between about 5 to about 20 amino acids in length, or between about 5 to about 15 amino acids in length. Alternatively, the peptide fragment may be between about 5 to about 10 amino acids in length.

Relaxin polypeptides modified at the N- and/or C-terminus by the addition, deletion or substitution of one or more amino acid residues as described above also fall within the scope of the present invention.

In accordance with the present disclosure modified relaxin polypeptides may be produced using standard techniques of recombinant DNA and molecular biology that are well known to those skilled in the art. Guidance may be obtained, for example, from standard texts such as Sambrook et al., Molecular Cloning: A Laboratory Manual, Cold Spring Harbor, N.Y., 1989 and Ausubel et al., Current Protocols in Molecular Biology, Greene Publ. Assoc. and Wiley-Intersciences, 1992. Methods described in Morton et al., 2000 (Immunol Cell Biol 78:603-607), Ryan et al., 1995 (J Biol Chem 270:22037-22043) and Johnson et al., 2005 (J Biol Chem 280:4037-4047) are examples of suitable purification methods for relaxin polypeptides, although the skilled addressee will appreciate that the present invention is not limited by the method of purification or production used and any other method may be used to produce relaxin for use in accordance with the methods and compositions of the present disclosure. Relaxin peptide fragments may be produced by digestion of a polypeptide with one or more proteinases such as endoLys-C, endoArg-C, endoGlu-C and staphylococcus V8-protease. The digested peptide fragments can be purified by, for example, high performance liquid chromatographic (HPLC) techniques.

The purification of modified relaxin polypeptides of the present disclosure may be scaled-up for large-scale production purposes. For this purpose a range of techniques well known to those skilled in the art are available.

Modified relaxin polypeptides of the present disclosure, as well as fragments and variants thereof, may also be synthesised by standard methods of liquid or solid phase chemistry well known to those of ordinary skill in the art. For example such molecules may be synthesised following the solid phase chemistry procedures of Steward and Young (Steward, J. M. & Young, J. D., Solid Phase Peptide Synthesis. (2nd Edn.) Pierce Chemical Co., Illinois, USA (1984).

In general, such a synthesis method comprises the sequential addition of one or more amino acids or suitably protected amino acids to a growing peptide chain. Typically, either the amino or carboxyl group of the first amino acid is protected by a suitable protecting group. The protected amino acid is then either attached to an inert solid support or utilised in solution by adding the next amino acid in the sequence having the complimentary (amino or carboxyl) group suitably protected and under conditions suitable for forming the amide linkage. The protecting group is then removed from this newly added amino acid residue and the next (protected) amino acid is added, and so forth. After all the desired amino acids have been linked, any remaining protecting groups, and if necessary any solid support, is removed sequentially or concurrently to produce the final polypeptide.

Embodiments of the present disclosure also provide isolated polynucleotides encoding relaxin polypeptides as described above, and variants and fragments of such polynucleotides.

Those skilled in the art will appreciate that heterologous expression of polypeptides may be improved by optimising the codons for the particular species in which the relaxin polypeptide is to be expressed. Accordingly, polynucleotides encoding relaxin polypeptides of the present disclosure may be codon-optimised for expression in a particular species.

Fragments of polynucleotides of the invention are also contemplated. The term “fragment” refers to a nucleic acid molecule that encodes a constituent or is a constituent of a polynucleotide of the invention. Fragments of a polynucleotide, do not necessarily need to encode polypeptides which retain biological activity. Rather the fragment may, for example, be useful as a hybridization probe or PCR primer. The fragment may be derived from a polynucleotide of the invention or alternatively may be synthesized by some other means, for example chemical synthesis. Polynucleotides of the invention and fragments thereof may also be used in the production of antisense molecules using techniques known to those skilled in the art.

In particular embodiments, polynucleotides of the present disclosure may be cloned into a vector. The vector may be a plasmid vector, a viral vector, or any other suitable vehicle adapted for the insertion of foreign sequences, their introduction into eukaryotic cells and the expression of the introduced sequences. Typically the vector is a eukaryotic expression vector and may include expression control and processing sequences such as a promoter, an enhancer, ribosome binding sites, polyadenylation signals and transcription termination sequences.

The present disclosure also provides antibodies that selectively bind to the modified relaxin polypeptides of the disclosure, as well as fragments and analogues thereof. Suitable antibodies include, but are not limited to polyclonal, monoclonal, chimeric, humanised, single chain, Fab fragments, and an Fab expression library. Antibodies of the present invention may act as agonists or antagonists of relaxin polypeptides, or fragments or analogues thereof.

Methods for the generation of suitable antibodies will be readily appreciated by those skilled in the art. For example, an anti-relaxin monoclonal antibody, typically containing Fab portions, may be prepared using the hybridoma technology described in Antibodies—A Laboratory Manual, Harlow and Lane, eds., Cold Spring Harbor Laboratory, N.Y. (1988).

Screening for the desired antibody can also be accomplished by a variety of techniques known in the art. Assays for immunospecific binding of antibodies may include, but are not limited to, radioimmunoassays, ELISAs (enzyme-linked immunosorbent assay), sandwich immunoassays, immunoradiometric assays, gel diffusion precipitation reactions, immunodiffusion assays, in situ immunoassays, Western blots, precipitation reactions, agglutination assays, complement fixation assays, immunofluorescence assays, protein A assays, and immunoelectrophoresis assays, and the like (see, for example, Ausubel et al., eds, 1994, Current Protocols in Molecular Biology, Vol. 1, John Wiley & Sons, Inc., New York). Antibody binding may be detected by virtue of a detectable label on the primary anti-relaxin antibody. Alternatively, the anti-relaxin antibody may be detected by virtue of its binding with a secondary antibody or reagent which is appropriately labelled. A variety of methods are known in the art for detecting binding in an immunoassay and are within the scope of the present invention.

Relaxin polypeptides and polynucleotides of the present disclosure may be useful as therapeutic agents. For example, modified polypeptides of the disclosure, polynucleotides encoding the same and compositions comprising such polypeptides or polynucleotides may be used in the treatment or prophylaxis of cardiovascular, fibrotic, renal and neurological diseases or other diseases or conditions associated with aberrant relaxin expression or activity. In exemplary embodiments the polypeptide is an agonist of a relaxin receptor, typically the RXFP3 receptor, and the disease or condition may be selected from, or is associated with, vascular disease including coronary artery disease, peripheral vascular disease, vasospasm including Raynaud's phenomenon, microvascular disease involving the central and peripheral nervous system, kidney, eye and other organs; treatment of arterial hypertension; diseases related to uncontrolled or abnormal collagen or fibronectin formation such as fibrotic disorders (including fibrosis of lung, heart and cardiovascular system, kidney and genitourinary tract, gastrointestinal system, cutaneous, rheumatologic and hepatobiliary systems); kidney disease associated with vascular disease, interstitial fibrosis, glomerulosclerosis, or other kidney disorders; psychiatric disorders including circadian/sleep disorders in adolescents, adults and the aged, including insomnia; anxiety states including panic attack, agoraphobia, global anxiety, phobic states, post-traumatic stress disorder; depression or depressive disorders including major depression, dysthymia, bipolar and unipolar depression and depressive symptoms of the neurodegenerative diseases; neurologic or neurodegenerative diseases (including memory loss or other memory disorders, dementias, Alzheimer's disease); neurodevelopmental disorders of Autism and Autism Spectrum Disorders (ASD), disorders of learning, attention and motivation (including Attention Deficit Hyperactivity Disorder, Tourette's disease, impulsivity, obsessive compulsive disorders, antisocial and personality disorders, negative symptoms of psychoses including those due to schizophrenia, acquired brain damage and frontal lobe lesions); addictive disorders (including drug, alcohol and nicotine addiction); movement and locomotor disorders (including Parkinson's disease, Huntington's disease and their depressive and cognitive symptoms, and motor deficits after stoke, head injury, surgery, tumour or spinal cord injury); immunological disorders (including immune deficiency states, haematological and reticuloendothelial malignancy; breast disorders (including fibrocystic disease, impaired lactation, and cancer); endometrial disorders including infertility due to impaired implantation; endocrine disorders (including adrenal, ovarian and testicular disorders related to steroid or peptide hormone production); delayed onset of labour, impaired cervical ripening, and prevention of prolonged labour due to fetal dystocia; sinus bradycardia; hair loss, alopecia; disorders of water balance including impaired or inappropriate secretion of vasopressin; and placental insufficiency.

In further exemplary embodiments the polypeptide is an antagonist of a relaxin receptor, typically the RXFP3 receptor, and the disease or condition is selected from, or is associated with, substance use, abuse and/or addiction, addictive behaviour and symptoms and conditions associated with substance abuse and addiction, Attention Deficit Hyperactivity Disorder (ADHD), obsessive compulsory disorder, autism and Autism Spectrum Disorders (ASD), neuroendocrine disorders of pregnancy and post-partum period (postnatal depression), and medication-related hyperactivity or hyper-arousal conditions.

Thus, in particular embodiments, modified relaxin polypeptides of the present invention that are antagonists of the RXFP3 receptor, such as the exemplified analogue 16 polypeptide, may be used in the prevention or inhibition of substance use, abuse and/or addiction, addictive behaviour, or a symptom, behaviour or condition associated with substance abuse and/or addiction. Exemplary addictive substances to which embodiments of the disclosure relate include, but are not limited to alcohol, opiates, cannabinoids, nicotine, inhalants and psychostimulants such as cocaine, amphetamine and methamphetamine. In particular embodiments the substance is selected from alcohol and opiates. Addiction to substances such as alcohol, opiates, cannabinoids, nicotine and psychostimulants is typically associated with a number of adverse or negative behaviours exhibited by addicts, which behaviours may serve to exacerbate, prolong or induce relapse into use or abuse of the substance, reinforce or exacerbate the addiction, or induce relapse into addiction and addictive behaviour patterns. Embodiments of the present disclosure provide methods and compositions for the prevention and inhibition of such negative behaviours including, but not limited to, the desire to consume the substance and substance-seeking behaviour. Other examples of negative behaviours associated with substance use or addiction include anxiety, dysphoria, stress reactivity and cue reactivity. Embodiments disclosed herein also provide for the treatment of the substance abuse or addiction.

In particular exemplary embodiments, polypeptides of the invention are useful as anxiolytic and/or antidepressant agents. As exemplified herein administration of the modified polypeptide designated analogue 15, comprising an A chain of SEQ ID NO: 4 and a B chain of SEQ ID NO: 2, decreases anxiety-like behaviours of adult rats in a light-dark box and elevated plus maze. Analogue 15 also decreases immobility in the forced swim test in rats that had previously undergone tests of anxiety-like behaviour. Those skilled in the art will appreciate that tests such as those exemplified herein are routinely used in the art, for example by the pharmaceutical industry, in screening compounds for potential therapeutic activity, such as the forced swim test in the case of compounds being evaluated for anti-depressant activity.

Antibodies to the modified relaxin polypeptides of the present disclosure may also be useful as therapeutic agents.

Where modified polypeptides of the present disclosure are agonists of the RXFP3 receptor, these molecules (and polynucleotides encoding same) find application, for example, in treating or preventing a disease or condition in a subject, by administering a therapeutically effective amount of such a molecule to the subject. Antagonists of the RXFP3 receptor as disclosed herein may also be useful as therapeutic agents where the inhibition of the activity or signalling from the RXFP3 receptor is desirable.

Modified polypeptides of the disclosure, and antibodies to such polypeptides, may also be employed as tools for the study of relaxin-3 biological activities.

In general, suitable compositions for use in accordance with the methods of the present modified polypeptides of the disclosure may be prepared according to methods and procedures that are known to those of ordinary skill in the art and accordingly may include a pharmaceutically acceptable carrier, diluent and/or adjuvant.

Compositions may be administered by standard routes. In general, the compositions may be administered by the parenteral (e.g., intravenous, intraspinal, intracerebroventricular, intranasal, subcutaneous or intramuscular), oral or topical route. Administration may be systemic, regional or local. The particular route of administration to be used in any given circumstance will depend on a number of factors, including the nature of the condition to be treated, the severity and extent of the condition, the required dosage of the particular compound to be delivered and the potential side-effects of the compound.

In general, suitable compositions may be prepared according to methods which are known to those of ordinary skill in the art and may include a pharmaceutically acceptable diluent, adjuvant and/or excipient. The diluents, adjuvants and excipients must be “acceptable” in terms of being compatible with the other ingredients of the composition, and not deleterious to the recipient thereof.

Examples of pharmaceutically acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

Compositions may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, in a form suitable for parenteral administration, e.g., intravenous, intraspinal, intracerebroventricular, intranasal, subcutaneous or intramuscular.

For administration as an injectable solution or suspension, non-toxic parenterally acceptable diluents or carriers can include, Ringer's solution, isotonic saline, phosphate buffered saline, ethanol and 1,2 propylene glycol. In some embodiments cerebrospinal fluid (CSF) may be used as a carrier.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Adjuvants typically include emollients, emulsifiers, thickening agents, preservatives, bactericides and buffering agents.

Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference herein.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The compositions may also be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which is incorporated herein by reference.

For the purposes of the present disclosure molecules and agents may be administered to subjects as compositions either therapeutically or preventively. In a therapeutic application, compositions are administered to a patient already suffering from a disease, disorder or condition, in an amount sufficient to cure or at least partially arrest the disease, disorder or condition and its complications. The composition should provide a quantity of the molecule or agent sufficient to effectively treat the patient.

The therapeutically effective dose level for any particular patient will depend upon a variety of factors including: the disorder being treated and the severity of the disorder; activity of the molecule or agent employed; the composition employed; the age, body weight, general health, sex and diet of the patient; the time of administration; the route of administration; the rate of sequestration of the molecule or agent; the duration of the treatment; drugs used in combination or coincidental with the treatment, together with other related factors well known in medicine.

One skilled in the art would be able, by routine experimentation, to determine an effective, non-toxic amount of agent or compound which would be required to treat applicable diseases and conditions.

Generally, an effective dosage is expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage may be up to about 500 mg/m². Generally, an effective dosage is expected to be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

Typically, in therapeutic applications, the treatment would be for the duration of the disease state.

Further, it will be apparent to one of ordinary skill in the art that the optimal quantity and spacing of individual dosages will be determined by the nature and extent of the disease state being treated, the form, route and site of administration, and the nature of the particular individual being treated. Also, such optimum conditions can be determined by conventional techniques.

It will also be apparent to one of ordinary skill in the art that the optimal course of treatment, such as, the number of doses of the composition given per day for a defined number of days, can be ascertained by those skilled in the art using conventional course of treatment determination tests.

Embodiments of the present disclosure also contemplate the administration of a polynucleotide encoding a modified relaxin polypeptide of the disclosure. In such situations the polynucleotide is typically operably linked to a promoter such that the appropriate polypeptide sequence is produced following administration of the polynucleotide to the subject. The polynucleotide may be administered to subjects in a vector. The vector may be a plasmid vector, a viral vector, or any other suitable vehicle adapted for the insertion of foreign sequences, their introduction into eukaryotic cells and the expression of the introduced sequences. Typically the vector is a eukaryotic expression vector and may include expression control and processing sequences such as a promoter, an enhancer, ribosome binding sites, polyadenylation signals and transcription termination sequences. The nucleic acid construct to be administered may comprise naked DNA or may be in the form of a composition, together with one or more pharmaceutically acceptable carriers.

Those skilled in the art will appreciate that in accordance with the methods of the present disclosure modified relaxin polypeptides may be administered alone or in conjunction with one or more additional agents. Additionally, the present disclosure contemplates combination therapy using modified relaxin polypeptides disclosed herein in conjunction with other therapeutic approaches to the treatment of diseases and disorders. For such combination therapies, each component of the combination therapy may be administered at the same time, or sequentially in any order, or at different times, so as to provide the desired effect. Alternatively, the components may be formulated together in a single dosage unit as a combination product. When administered separately, it may be preferred for the components to be administered by the same route of administration, although it is not necessary for this to be so.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be described with reference to the following specific examples, which should not be construed as in any way limiting the scope of the invention.

### EXAMPLES

### General Methods

### Animals

Experiments were conducted with the approval of the Florey Neuroscience Institutes Animal Ethics Committee and according to the guidelines issued by the National Health and Medical Research Council of Australia. All efforts were made to minimise the number of animals used. Male Sprague-Dawley rats supplied by the Animal Resources Centre, Perth, Wash., Australia, weighed 250-300 g on arrival at the Florey Neuroscience Institutes. Rats were single-housed under ambient conditions (21° C.) and maintained on a 12 h light:dark cycle (lights on 0700-1900) with access to food (laboratory chow) and water ad libitum. Rats were acclimatised to the animal facility for at least 1 week prior to any experimentation.

Stereotaxic Implantation of a Guide Cannula into Lateral Ventricle

Each rat was deeply anaesthetised with 4% isoflurane in room air, 2 L/min (Delvet, Seven Hills, NSW, Australia) and maintained with ˜2% isoflurane in room air, 0.2 L/min. The head was positioned in a stereotaxic frame (David Kopf Instruments, Tujunga, Calif., USA) and a small incision was made in the skin. The area was cleaned and dried, and a small hole was drilled in the skull, through which a stainless-steel guide cannula (22 gauge, cut 5 mm below pedestal; Plastics One, Roanoke, Va., USA) was implanted using the following coordinates relative to Bregma: anteroposterior, −0.8 mm; mediolateral, −1.5 mm; dorsolateral −3.5 mm (Paxinos and Watson 2007). Three screws (1.4 mm diameter×3 mm length; Mr Specs, Mordialloc, VIC, Australia) were attached to the skull, and the cannula was affixed to the screws and the skull using dental cement (Vertex-Dental, Zeist, The Netherlands). A 30-gauge stylet was inserted into the cannula to maintain patency.

After surgery each rat was placed under a heat lamp until regaining consciousness and housed individually in clean cages. Meloxicam (3 mg/kg, i.p; Troy Laboratories, Smithfield, NSW, Australia) was administered to provide acute post-operative analgesia; and 0.5 mg/ml paracetamol in 5% sucrose/water (Panadol® Rapid Soluble, GlaxoSmithKline, Ermington, NSW, Australia) was given for 3 days as ongoing post-operative analgesia. An antibiotic injection was also administered peri-operatively (5 mg enrofloxacin, Bayer Australia Ltd, Pymble, NSW, Australia). Rats were single-housed and allowed to recover for 7 days, during which time they were handled and weighed daily to habituate them to the experimenter.

### Intracerebroventricular Infusions and Verification of Cannula Location and Patency

Lateral ventricle infusions of peptides or vehicle were made using 29-gauge hypodermic tubing (Small Parts Inc., Miramar, Fla., USA) connected to a 10-μl microsyringe (Hamilton Instruments, Reno, Nev., USA) by polyethylene tubing (0.80 mm outer and 0.40 mm internal diameter; Microtube Extrusions, North Rocks, NSW, Australia). Rats were gently held in a towel, and the injector, was inserted into the protruding guide cannula. Infusions of 5 μl were delivered manually over the course of ˜20 s, with care to ensure that all the solution was delivered. The injector was left in place for ˜10 after infusion. 5 μg analogue 15 (˜1.1 nmol) in 5 μl was injected into the ‘agonist-treated’ group of rats. ‘Control’ rats received 5 μl aCSF.

Correct cannula positioning was verified in each rat by testing the acute dipsogenic response to an injection of angiotensin II (5 μl of a 4 ng/μl solution; Auspep, Parkville, VIC, Australia) in artificial cerebrospinal fluid (aCSF; 1470 mM NaCl; 40 mM KCl; 8.5 mM MgCl₂; 23 mM CaCl₂). Dipsogenesis was defined as repeated drinking episodes of 5 sec that commenced within 1 min of angiotensin II administration. The dipsogenic response to angiotensin II was re-tested after each behavioural test and the behavioural data in response to analogue 15 injection were excluded if the rat did not display a positive angiotensin II elicited drinking response.

Data analysis and generation of histograms were routinely performed using GraphPad Prism Version 5.00 for Windows (GraphPad Software, San Diego, Calif., USA). Results are expressed as mean±SEM. Statistical significance was routinely evaluated using a Student's t-test, unless otherwise stated. Where necessary, data was analysed by two-way ANOVA with a Bonferroni post-hoc test (SigmaStat Version 2.03, Aspire Software International, Ashburn, Va., USA). If any data point was standard deviations from the remaining data points, it was excluded as an outlier.

### Example 1

### Modified Relaxin-3 Polypeptide Construction

Synthetic modified relaxin-3 polypeptides were generated by solid phase peptide synthesis.

Native relaxin-3 not only binds and activates RXFP3, but also the relaxin-2 cognate receptor RXFP1. The present inventors sought to produce modified relaxin-3 polypeptides that are selective for RXFP3. A previous study has demonstrated that a relaxin-3 polypeptide with a truncation of 10 amino acids from the N-terminus of the H3 relaxin A chain retains the ability to bind and activate RXFP1 (Hossain et al., 2008).

The inventors first generated a modified relaxin-3 polypeptide, designated herein analogue 14. Analogue 14 comprises a full length native H3 relaxin B chain sequence (comprising the amino acid sequence set forth in SEQ ID NO.2) and a full length H3 A chain, however the A chain comprises two amino acid substitutions, at positions 10 and 15 of the H3 relaxin A chain sequence set forth in SEQ ID NO.1, wherein cysteine residues are replaced by alanine residues by site-directed mutagenesis. The amino acid sequence of the modified A chain of analogue 14 is set forth in SEQ ID NO.3. The cysteine residues at positions 10 and 15 of the H3 relaxin A chain are responsible for the intra-chain disulphide bond, and thus analogue 14 contains no intra-chain disulphide bonds within the A chain, however retains the two native inter-chain disulphide bonds.

From this modified A chain of analogue 14, the inventors then truncated the N-terminus by 10 amino acids resulting in the amino acid sequence set forth in SEQ ID NO.4. The resulting modified relaxin-3 polypeptide (analogue 15) comprises the A chain having the amino acid sequence of SEQ ID NO.4 and the native H3 B chain sequence of SEQ ID NO.2. Thus, the two inter-chain disulphide bonds remain intact but, as with analogue 14, there is no intra A chain disulphide bond. Analogue 15 is also referred to as [R3A(11-24,C15→A)B], RXFP3-selective agonist, analogue 2, or RXFP3-A2. Analogue 15 was synthesized using solid phase peptide synthesis and purified using reverse phase HPLC. The identity and purity of the peptide was confirmed by reverse phase HPLC, MALDI-TOF mass spectrometry and NMR analysis and the amino acid composition was checked. From the B chain of analogue 15, the inventors then truncated the N-terminus by 5 amino acids resulting in the amino acid sequence set forth in SEQ ID NO:6. The resulting modified relaxin-3 polypeptide (analogue 17) comprises the A chain from analogue 15, having the amino acid sequence of SEQ ID NO.4, and the B chain having the amino acid sequence of SEQ ID NO.6. Analogues 18 and 19 were generated by truncating the B chain present in analogue 17 by 2 residues (ΔGV) and 4 residues (ΔGVRL), respectively, from the N-terminus. The B chain amino acid sequence of analogue 18 is shown in SEQ ID NO:7. The B chain amino acid sequence of analogue 19 is shown in SEQ ID NO:8.

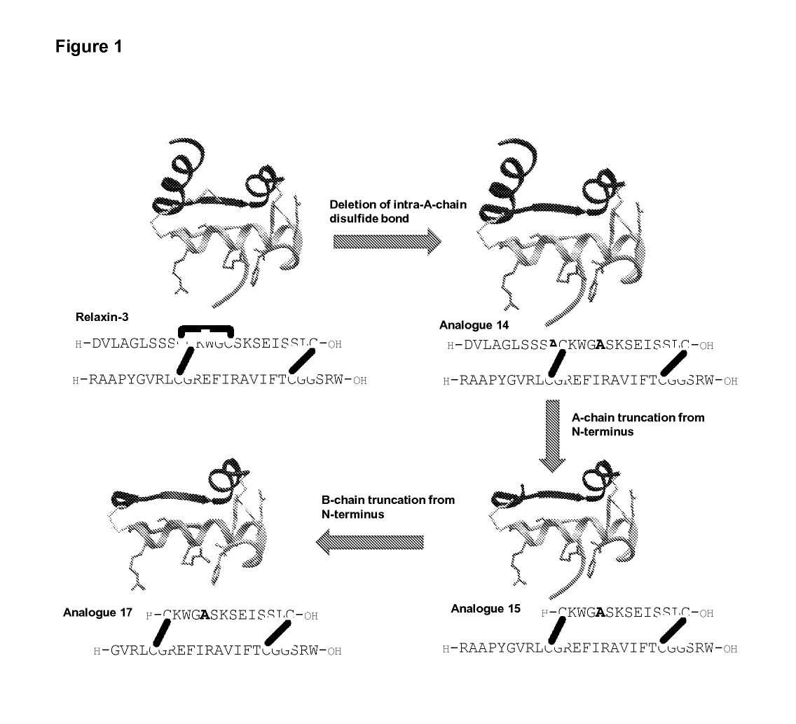

The primary sequences and predicted tertiary structures for analogues 14, 15 and 17 are shown in FIG. 1.

The peptides were synthesised using pre-loaded Fmoc-Trp(Boc)-NovaSyn®TGA and Fmoc-Cys(Acm)-Ac-S-TentaGel. The resin-bound peptides were cleaved using a mixture of TFA:DoDt:H₂O:TIPS (94:2.5:2.5:1). The chain combination between A- and B-chain was carried out by first converting the A-chain Cys(tBu)¹⁰ to Cys(SPy) using TFMSA to deprotect the tert-butyl group and DPDS to convert it to 2-pyridylsulfenyl derivative. The second inter-chain disulfide bond was formed using 20 mM iodine in acetic acid. All crude peptides were analysed and purified by RP-HPLC using, Waters XBridge™ columns (4.6×250 mm, C18, 5 μm) and (19×150 mm, C18, 5 μm), respectively. Peptides were each characterised using MALDI-TOF/TOF mass spectrometry (Bruker Daltonics, Germany).

### Example 2

### Biological Activities of Modified Relaxin-3 Polypeptides

Inhibition of Forskolin-Induced cAMP Accumulation in RXFP3 Expressing CHO-K1 Cells:

The potency of the modified relaxin-3 polypeptides analogue 14, analogue 15 and analogue 17 was assessed by measuring their influence on forskolin-induced cAMP signalling in CHO-K1 cells stably expressing RXFP3 using a cAMP reporter gene assay. CHO-K1 cells were subcultured in clear 96 well plates (10,000 cells/well/200 μl media) and after 24 h, they were co-transfected with pCRE-8-galactosidase reporter plasmid. 24 hours later, cells were treated with 5 μM forskolin together with increasing concentration of relaxin-3 polypeptides. 100 nM of H3 relaxin was used for maximal stimulation and the untreated cells were used as controls. The stimulation was carried out for 6 h after which the media was aspirated and the cells were frozen at −80° C. overnight. The cells were thawed to room temperature and the amount of cAMP-induced β-galactosidase expression was measured by adding 25 μl of 0.1× buffer A (100 mM Na₂HPO₄ (pH 8.0), 2 mM MgSO₄, 0.1 mM MnCl₂) and shaking at room temperature for 10 minutes to hypotonically lyse the cells. Then 100 μl of buffer B (similar to buffer A with additional 0.5% Triton X-100 and 40 mM β-mercaptoethanol) was added to each well with shaking for 10 minutes at room temperature. Finally, 25 μl of substrate for 6-galactosidase (chlorophenol red β-D galactopyranoside) was added to each well with shaking until the colour change was observed. The absorbance was measured at 570 nm on Victor³ plate reader (PerkinElmer, Glen Waverly, VIC). The data were analysed using GraphPad PRISM 4 (GraphPad Inc., San Diego, USA). The inhibition by H3 relaxin and analogues 14, 15 and 17 of forskolin-induced cAMP accumulation curves was fitted to a single-site sigmoidal dose-response curve with variable slope. Statistical analysis was carried out using one-way ANOVA with Bonferroni's multiple comparison test.

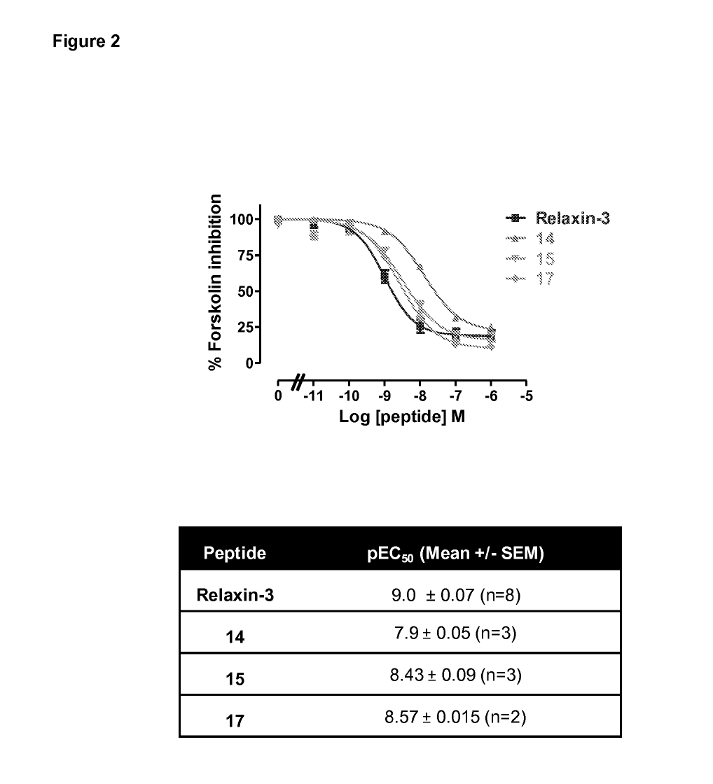

As shown in FIG. 2, analogues 14, 15 and 17 each display essentially the same activity as H3 relaxin at the RXFP3 receptor. Similar results to those obtained for analogue 14 were obtained for analogues 18 and 19 (data not shown).

Stimulation of cAMP Accumulation in RXFP1 Expressing HEK 293-Cells

The cAMP assay was carried out by adding increasing concentration of polypeptides in cell culture media to HEK-293 cells expressing RXFP1. The cells were incubated with the polypeptides for 6 hours at 37° C. in 96-well plates. Each concentration point was performed in triplicate and the data expressed as the mean±SEM of three independent experiments. The data were analysed using GraphPad PRISM 4 (GraphPad Inc., San Diego, USA).

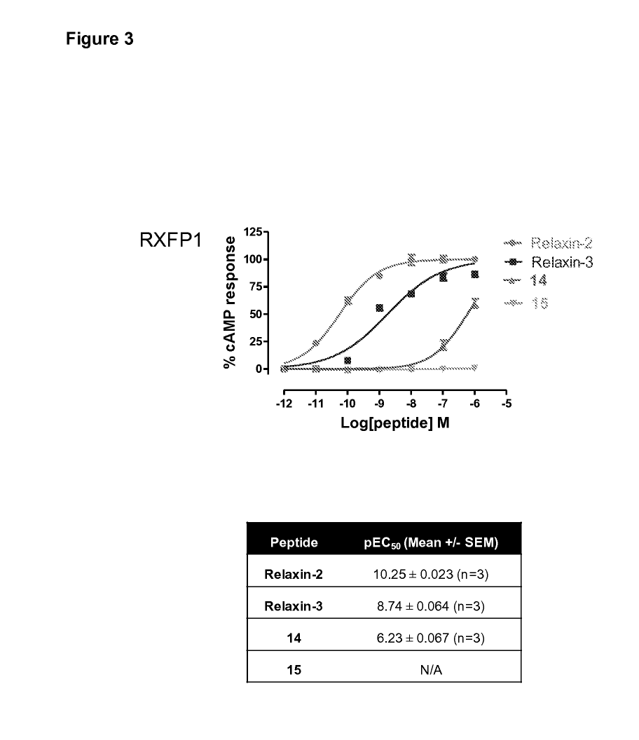

As shown in FIG. 3, native H3 relaxin activates RXFP1, as measured by cAMP response, to a similar extent to relaxin-2. In contrast, the activity of analogue 14 at RXFP1 is markedly reduced and more significantly, analogue 15 displays no activity at RXFP1. Similar results were obtained with analogues 18 and 19 (data not shown).

### Receptor Binding Affinities

Competition binding assays were used to determine the binding affinity of the analogues at RXFP1, RXFP3 and RXFP4, using europium-labelled H2 relaxin for RXFP1, europium-labelled H3 relaxin B-chain/INSL5 A-chain chimeric peptide for RXFP3 and europium-labelled mouse INSL5 for RXFP4. HEK-293T cells stably expressing RXFP1 and CHO-K1 cells stably expressing RXFP3 and RXFP4 were plated into a 96 well plate (Viewplate; opaque white wall and clear bottom, PerkinElmer, Glen Waverly, Vic, Australia) at a density of 5×10⁴ cells/well and grown over night to reach ˜90% confluence before experimentation. Binding assays were conducted as described in Shabanpoor et al., 2008, Bioconjug Chem, 19:1456-1463. Briefly, the competition binding assay was done using a single concentration of Eu-labelled INSL5A/H3 relaxinB (0.5 nM), Eu-labelled H2 relaxin (1 nM) and Eu-labelled mouse INSL5 (2.5 nM) in the presence of increasing concentrations of H3 relaxin analogues in comparison to the native receptor ligands. Each concentration point was performed in triplicate and the data expressed as the mean±SEM of three independent experiments.

Results of competition binding assays are shown in Table 1. Analogue 15 demonstrated a binding affinity very similar to H3 relaxin. Analogues 14 and 17-19 each displayed similar binding affinity for RXFP3 albeit lower than H3 relaxin and analogue 15. Importantly none of the analogues had any affinity for RXFP1 in contrast to native H3 relaxin. Analogue 15 had similar binding affinity to native INSL5 for RXFP4.

TABLE 1
Receptor binding affinity (pKᵢ) and activation potency (pEC₅₀) of human H3 relaxin and
analogues at RXFP1, RXFP3 and RXFP4

| | pKᵢ (M) | | pIC₅₀ (M) | pEC₅₀ (M) | | |
|---|---|---|---|---|---|---|
| Peptide | RXFP3 | RXFP1 | RXFP4 | RXFP3 | RXFP1 | RXFP4 |
| H3 relaxin | 7.78 ± 0.06 | 8.6 ± 0.01 | ND | 9.0 ± 0.07 | 8.74 ± 0.06\hat{ } | 8.94 ± 0.13 |
| hINSL5 | ND | ND | 7.33 ± 0.08 | ND | ND | 8.51 ± 0.11 |
| 14 | 7.3 ± 0.12** | <6 | ND | 7.9 ± 0.05# | 6.23 ± 0.07\hat{ } | ND |
| 15 | 7.87 ± 0.12* | <5 | 7.1 ± 0.07 | 8.43 ± 0.09 | N/A | 7.7 ± 0.07§ |
| 17 | 6.95 ± 0.03** | <5 | ND | 8.37 ± 0.10 | N/A | 7.85 ± 0.16§ |
| 18 | 6.95 ± 0.13** | <5 | ND | 8.29 ± 0.11# | N/A | 8.1 ± 0.21§ |
| 19 | 6.96 ± 0.11** | <5 | ND | 7.9 ± 0.02# | N/A | 8.46 ± 0.17 |

n = 3-9 independent experiments.
N/A: no activity,
ND; not determined,
**p < 0.05 vs H3 relaxin,
*p > 0.05 vs H3 relaxin,
#p < 0.05 vs H3 relaxin, analogues 15 and 17,
§p < 0.05 vs hINSL5 and analogue 19,
\hat{ }p < 0.05, “p < 0.05 vs hINSL5 and analogue 15.

The activities of the various polypeptides at RXFP3 (FIG. 2) and RXFP1 (FIG. 3) clearly demonstrate that the modified relaxin-3 polypeptide analogue 14 displays increased selectivity for the RXFP3 receptor over RXFP1, whereas analogues 15, 17, 18 and 19 are completely selective for RXFP3 over RXFP1. Thus relaxin-3 intra-A-chain disulphide bond removal is not sufficient to impart strong selectivity (or specificity) for RXFP3 as its deletion can still result in a polypeptide able to activate RXFP1 at micromolar concentrations. However deletion of 10 residues from the N-terminus of an A-chain lacking intra-chain disulphide bonds can completely abolish the activity of a relaxin-3 polypeptide at RXFP1 without any loss of potency at RXFP3.

### TGF-β1-Induced Collagen Expression in Human Dermal Fibroblasts

Based on the inventors recent observations that H2 and H3 relaxin inhibit TGF-β1-stimulated collagen deposition from rat ventricular fibroblasts in vitro, which only express RXFP1 but not RXFP3 (Hossain et al., 2011, Biochemistry 50:1368-1375); the effects of analogue 15 vs H2 and H3 relaxin were evaluated in TGF-β1-stimulated human dermal fibroblasts (BJ3 cells; kindly provided by Associate Professor William Hahn; Dana-Farber Cancer Institute, Harvard Medical School, Boston, Mass.); which also express RXFP1. BJ3 cells were plated at a density of 1×10⁶/well in 6-well plates and either untreated or treated with TGF-β1 (2 ng/mLl; R&D Systems; Minneapolis, Minn.) alone; TGF-β1 (2 ng/mL) plus H2 relaxin (100 ng/mL; 16.8 μM); TGF-β1 (2 ng/mL) plus H3 relaxin (100 ng/mL; 18.2 μM) or TGF-β1 (2 ng/mL) plus analogue 15 (100 ng/mLl; 22 μM) for 72 hr. The deposited collagen into the cell layer was hydrolysed and analysed for hydroxyproline content, as previously described (Samuel et al., 1996, Endocrinology 137:3884-3890). Hydroxyproline values were then converted to total collagen content by multiplying by a factor of 6.94 (based on hydroxyproline representing approximately 14.4% of the amino acid composition of collagen in most mammalian tissues). These experiments were performed 3-4 separate times in duplicate.

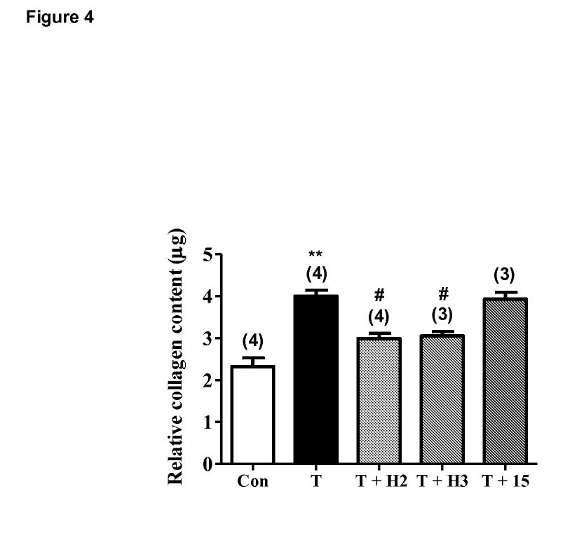

H2 and H3 relaxin comparably lowered the level of collagen by 35-40%, respectively, compared to TGF-β1 only treatment of cells. However, as anticipated from the RXFP1 binding, analogue 15 did not alter the level of TGF-β1-stimulated collagen expression (FIG. 4), further demonstrating its selectivity for RXFP3.

### ERK1/2 Phosphorylation in RXFP3 Expressing Cells

The ability of native H3 relaxin and analogue 15 to activate ERK1/2 kinase phosphorylation was determined. Stable CHO-RXFP3 cells were plated into 96-well plates (5×10⁴ cells/well) and grown overnight in DMEM/Ham's F-12 medium at 37° C., 5% CO₂. Cells were then washed twice with PBS and serum starved for 8 h before their stimulation with different concentrations of peptides, serum free DMEM/Ham's F-12 medium (vehicle control) or 10% FBS (positive control) for 5 min. Following treatment, cells were lysed according to the manufacturers instructions using 100 μL of lysis buffer and frozen at −20° C. For detection of ERK1/2 kinase phosphorylation, 4 μL of the thawed sample (cell lysate) was transferred to a white 384-well microplates (Proxiplates, PerkinElmer) and 5 μL of the combination buffer with AlphaScreen donor beads (40 parts reaction buffer, 10 part activation buffer and 1 part acceptor beads) was added. Plates were incubated for 2 h at 23° C. in the dark on an oscillating platform. Subsequently, 2 μL of the dilution buffer with AlphaScreen acceptor beads (20 parts dilution buffer, and 1 part donor beads) was added. Plates were again incubated for 2 h at 23° C. in the dark on the oscillating platform. The AlphaScreen signal (counts per second) was measured in 384-well microplates (Proxiplates) on an EnVision Multilabel Plate Reader (PerkinElmer) with excitation at 680 nm and emission at 520 to 620 nm. Both H3 relaxin and analogue 15 induced ERK1/2 phosphorylation in a concentration dependent manner with analogue 15 producing a slightly greater effect (pEC50 values of 9.83±0.14 (n=5) and 10.4±0.11 (n=4) respectively, p<0.05).

### Example 3

### Modified Relaxin-3 Polypeptide Antagonist of RXFP3

As described above, the relaxin-3 polypeptide designated herein as analogues 15 and 17 are highly selective agonists at RXFP3.

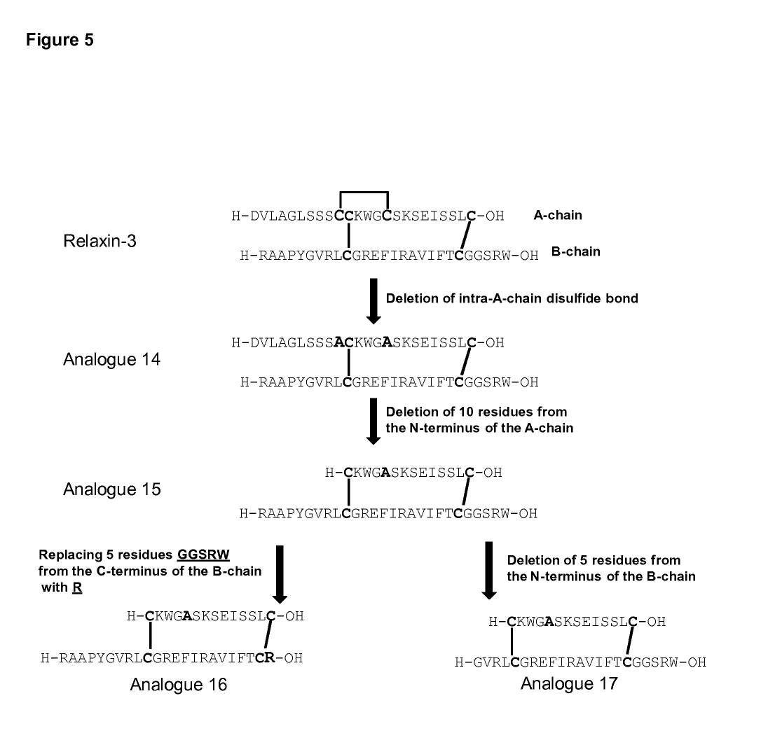

Analogue 15 was modified to generate a selective antagonist. This was achieved by deleting 5 residues from the C-terminus of the B-chain (G^{B23} G^{B24} S^{B25} R^{B26} W^{B27}) of analogue 15 and adding an arginine at position B²³ (FIG. 5). The resultant analogue is termed herein analogue 16.

Binding of analogue 16 to RXFP3 was determined using a whole cell receptor binding assay. CHO-K1 cells stably expressing RXFP3 were subcultured into a 96 well viewplate (opaque white wall and clear bottom, PerkinElmer, Glen Waverly, VIC) at a density of 50,000 cells/well/200 μl media 24 h before experimentation to reach ˜90% confluence. Cells were grown in DMEM/Hams F12 media supplemented with 5% (v/v) FCS, 2 mM L-glutamine, 100 μg/ml penicillin and 100 μg/ml streptomycin. The binding assay was conducted by making appropriate concentrations of Eu-labelled chimera peptide and relaxin-3 analogues in RXFP3 binding buffer (100 mM HEPES, 100 mM NaCl, 5 mM KCl, 1.3 mM MgSO₄, 15 mM NaOAc, 10 mM glucose and 1 mM EDTA; pH: 7.6) containing 1% BSA. The saturation binding experiment was carried out using increasing concentrations of Eu-DTPA-INSL5A/relaxin-3B in presence and absence of 1 μM relaxin-3. The competition binding assay was done using a single concentration (0.5 nM) of Eu-labelled INSL5A/relaxin-3B in presence of increasing concentration of relaxin-3 and its analogues. Each concentration point was performed in triplicate and the data expressed as the mean±SEM of three independent experiments. The data were analysed using GraphPad PRISM 4 (GraphPad Ins., San Diego, USA.

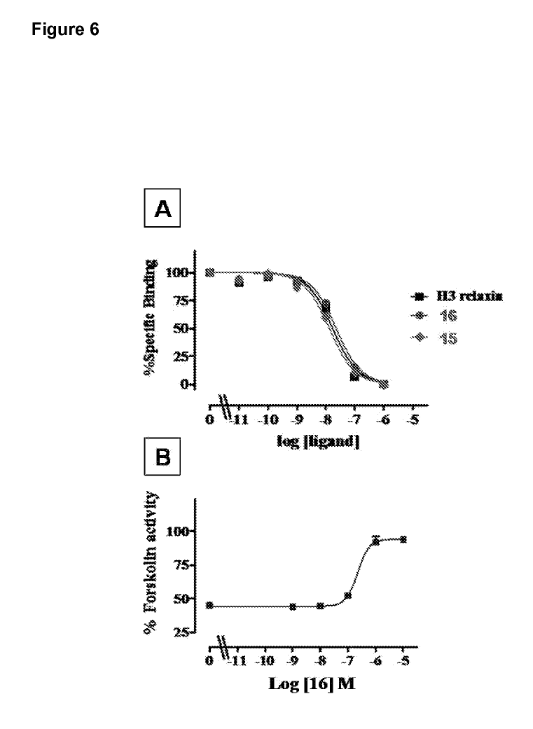

Analogue 16 showed similar receptor binding affinity as native relaxin-3 and analogue 15 against RXFP3 (FIG. 6A).

To determine the nature of the activity of analogue 16, its influence on forskolin-induced cAMP signalling in CHO-K1 cells stably expressing RXFP3 was determined using a cAMP reporter gene assay as described in Example 2. As shown in FIG. 6B, analogue 16 inhibited the cAMP activity of relaxin-3 in RXFP3 cells. Relaxin-3 at 10 nM concentration inhibited forskolin-induced cAMP production to about 50%, and the addition of increasing concentrations of analogue 16 rescued the level of cAMP back to about 100% by binding to RXFP3 and preventing its activation by relaxin-3. Thus analogue 16 is a selective antagonist of RXFP3.

The ability of analogue 16 to activate ERK1/2 kinase phosphorylation was determined using the method as described in Example 2. However in this instance cells were pre-treated with analogue 16 for 1 h prior to stimulation with H3 relaxin or analogue 15 for 5 min. Analogue 16 did not induce phosphorylation of ERK1/2. However, increasing concentration of analogue 16 antagonized ERK1/2 phosphorylation induced by H3 relaxin (10 nM) (data not shown). The ability of analogue 16 to inhibit H3 relaxin induced p38MAPK phosphorylation was also tested in the same way as ERK1/2 phosphorylation. Analogue 16 antagonised H3 relaxin-induced p38MAPK phosphorylation in a dose dependent manner (data not shown).

### Example 4

### Effect of Analogues 15 and 16 on Food Intake in Rats

Rats were habituated for a minimum of 7 days to the holding room and behavioural studies were performed during the light phase beginning at 1030-1100 h. Groups of rats were injected using a crossover design, whereby each rat received peptide and vehicle, with days between tests. Cannulated rats that had no drinking response were given ‘mock’ injections by inserting a stylet into their cannula and treated as controls (see analysis). Following the infusion, each rat was placed back into their home cage, where a pre-weighed amount of rat chow (11-14 g) was located in the food compartment of the wire cage lid. A pre-weighed water bottle was also placed in its usual compartment. Food and water were weighed at hourly intervals for up to 4 h post-injection, with minimal disturbance to the rat. For rats that received two injections 10 min apart, the pre-weighed rat chow was placed into the cage lid after the first injection, but the weighing of food and water was done at hourly intervals after the second injection.

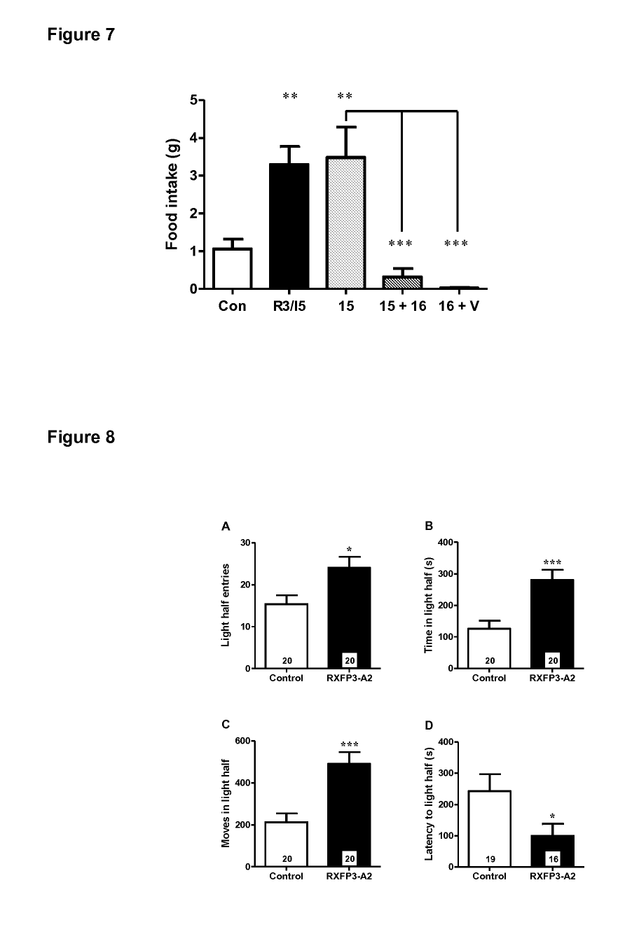

Central administration of analogue 15 (1.1 nmol, icy) to satiated rats during the early light phase significantly increased food intake within the first hour after injection, compared to control vehicle or mock injections (FIG. 7). The magnitude of the effect on food intake was comparable to that observed after administration of R3/15 (1 nmol, icy). Central administration of analogue 16 (4.8 nmol, icy) had no significant effect on food intake relative to control, but inhibited the effect of a subsequent icy injection of analogue 15 (1.1 nmol, icy). The amount of food consumed after the different treatments was: Control 1.07±0.25 g; analogue 15 3.48±0.81 g, P<0.01 vs control; R3/15 3.30±0.48 g, P<0.01 vs control; analogue 15+16 0.32±0.22 g, P<0.001 vs R3/15 and analogue 15; analogue 16 plus vehicle 0.029±0.017 g, P<0.001 vs R3/15 and analogue 16 (FIG. 7). None of the treatments had a significant effect on the amount of water consumed in conjunction with the stimulation of feeding (data not shown).

### Example 5

### Effect of Analogue 15 Administration on Rat Behaviour

The effect of modified relaxin polypeptide analogue 15 on rat behaviours associated with anxiety was tested by way of central administration of analogue 15 to the rat brain, and observation and quantification of subsequent behaviours in a variety of stressful situations. These behavioural tests were conducted during the light phase (0700-1900). Rats were habituated to the behavioural test room overnight prior to testing. Rats that underwent several anxiety-like tests had a minimum of 3 days separation between paradigms. Initially, rats were randomly assigned to peptide or vehicle groups, but where rats in a cohort underwent several tests, peptide and vehicle treatment was alternated in a counter-balanced manner. The majority of the behavioural data were generated by automated software analysis, and so were unaffected by any possible observer interference/bias. Tests that required scoring by an observer were conducted under blinded conditions.

### Example 5A

### Light-Dark Box

Rats were placed in the 42 cm (length)×42 cm (width)×40 cm (height) automated locomotor cell (Tru Scan Photobeam Rat Arena, E63-20; Coulbourn Instruments, Whitehall, Pa., USA). Half of the locomotor cell was covered by the ‘light-dark’ box made of plastic opaque to visible light, but transparent to photo beams. A small opening (7 cm×7 cm) enabled rats to enter or leave the ‘dark’ side. The light side was lit by an array of light-emitting diodes (400 lux in the centre) creating an aversive stimulus. Horizontal and vertical movements (rearing) of rats were tracked using Tru Scan 2.03 software (Coulbourn Instruments), which provided 5 min time bins and total session data outputs. The height for vertical plane (rearing) recordings was set at 19 cm. Rats received an icy injection 10 min prior to being placed in the dark side of the box at the beginning of the experiment and sessions lasted 20 min. Boxes were cleaned with warm water and dried between experiments.

Parameters measured included number of entries into the light compartment, time spent in light compartment, number of moves in the light compartment and latency to enter the light compartment (emergence). Rats that did not enter the light compartment at all were excluded from the ‘latency’ results.

FIG. 8 indicates that intracerebroventricular (icy) injection of 5 μg (˜1.1 nmol) of analogue 15 decreased anxiety-like behaviour in the light-dark box. Specifically, icy infusion of analogue 15 significantly increased the number of entries, time spent and number of moves in the light compartment, and decreased the latency to enter the light compartment.

There was a clear decrease in anxiety-like behaviour in rats treated with analogue 15 cf. the control group. Icy infusions of analogue 15 significantly altered several parameters measured in the light-dark box. The number of entries, the time spent, and number of moves in the light compartment, were increased, whereas the latency to enter the light compartment was reduced. There was no significant difference in the total number of moves (data not shown).

### Example 5B

### Elevated Plus Maze

The elevated plus maze consisted of four arms (44 cm long×12 cm wide) projecting from a central square (12 cm×12 cm), with a height of 72 cm from the ground. Two opposing arms had high 10 cm walls and were designated ‘closed’ arms. A small 0.6 cm ledge was present on either side of the ‘open’ arms to prevent rats falling off when they turned around. The apparatus was placed in the middle of the experimental room under low lux (˜50 lux closed arms, ˜70 lux open arms). Rats received any icy injection 10 min prior to being placed in the central square, facing an open arm, and were allowed to explore the apparatus for 10 min, while being recorded by EthoVision® software (EthoVision®, Version 3.0.15, Noldus Information Technology, Wageningen, The Netherlands).

Parameters measured included time spent in and entries into the open and closed arms. Centre and arms boundary thresholds were defined as all four limbs must enter an arm to be recorded (Featherby et al., 2008, Br J Pharmacol, 154: 417-428). The apparatus was cleaned and dried with 80% ethanol, followed by water, between experiments.

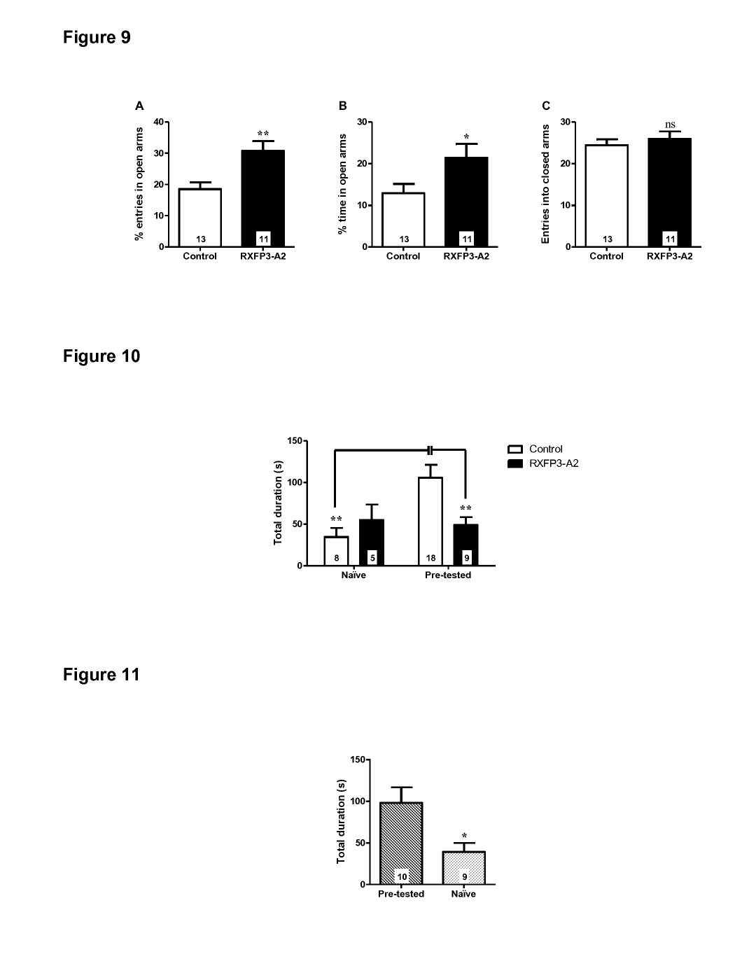

A separate cohort of rats underwent testing in the elevated plus maze. Icy injections of 5 μg (−1.1 nmol) analogue 15 decreased anxiety-like behaviour. Specifically, icy infusion of analogue 15 significantly increased the number of entries into the open arms and time spent in the open arms. There was no significant difference in the number of entries into the closed arms of the elevated plus maze (FIG. 9). The elevated plus maze test is based on the natural aversion of rodents for open spaces and reflects the conflict between exploration and aversion to elevated open places. Central injection of analogue 15 produced a clear anxiolytic effect in the elevated plus maze, including an increased percentage of open arms entries and time spent in the open arms, which are considered measures of anxiety-like behaviour (fellow et al., 1985, J Neurosci Methods 14: 149-167). There was no significant difference in the number of entries into the closed arms, which is considered a measure of motor activity in this test (Fernandes and File. 1996, Pharmacol. Biochem Behav 54: 31-40; File, 2001, Behav Brain Res 125: 151-157).

### Example 5C

### Repeat Forced Swim Test

Rats were placed in a tall glass vessel (21 cm×30 cm×51 cm with curved corners) filled with tap water to a height of 33 cm and a temperature between 22-24° C. Rats were kept in the water for 10 min before being removed and towelled dry, and placed under a heat lamp for about 30 min. The forced swim session was repeated at the same time the following day and rats received any icy injection 10 min prior to the start.

Rats were filmed using a Panasonic Colour CCTV Camera (Matsushita Electric Industrial Co Ltd, Osaka, Japan), and were manually scored using the EthoVision® program (Noldus Information Technology) for the total amount of time spent in the Porsolt (immobile) posture, the frequency of Porsolt posture, and latency to enter the Porsolt posture. The Porsolt posture was defined as immobility in all four limbs for ≦1 s. If the front paws moved to steady the rat during its immobile posture, this was not counted as a break in immobility.

Different cohorts of rats were used in this example. An initial cohort underwent multiple behavioural tests—firstly in the light-dark box, followed 4-5 days later by a large open field, then the repeat forced swim test, 13-14 days later. Another group (termed ‘experimentally naïve’) underwent testing only in the repeat forced swim test. Due to differences in the behaviour of control rats in the ‘experimentally naïve’ and ‘multiple-test’ cohorts in the repeat forced swim test, a separate cohort of rats was investigated. These uncannulated rats were divided into two groups: one underwent testing in the light-dark box and the large open field, prior to testing in the repeat forced swim test (over the same timeline as the previous cohort of ‘multiple-test’ rats) and another (‘experimentally naïve’) group had no testing prior to the repeat forced swim test, but was handled regularly (daily) prior to testing. A further cohort of rats underwent testing in the elevated plus maze.

Rats that had been tested in the light-dark box and large open field were rested for 13-14 days before exposure to the repeat forced swim test. No treatments were given on the first day of forced swimming. On the second day, acute icy injections were administered 10 min prior to the forced swim test. A separate cohort of rats was tested that were ‘experimentally naïve’ prior to the repeat forced swim test, i.e. had undergone no previous behavioural testing.

Comparison of the performance of the two cohorts of rats in the forced swim test using a two-way ANOVA (SigmaStat), comparing history (naïve versus pre-tested) and treatment (control versus analogue 15) as factors, demonstrated a significant interaction between history and treatment in the total duration in the immobility (Porsolt) posture (p=0.036). A Bonferroni post-hoc test revealed a significant difference between the naïve vs pre-tested rats within the control groups (p=0.002), and the control vs analogue 15 treated rats within the pre-tested group (p=0.009; FIG. 10). There were no significant differences in immobility frequency or latency to first immobility (data not shown).

In order to confirm that previous testing in anxiety-like paradigms and associated handling caused the increase in immobility during the forced swim test, a separate cohort of rats was tested. This cohort, which did not undergo icy cannulation, was divided into two groups: one group underwent testing in the both the light-dark box and the large open field prior to testing in the forced swim test, the other ‘experimentally naïve’ group had no behavioural testing prior to the forced swim test, but was handled regularly by the experimenters.

There was a significant difference between these two groups in immobility in the forced swim test, with a significantly decreased total duration of time spent in the Porsolt (immobile) posture in the ‘experimentally naïve’ group (FIG. 11). There was also a trend for decreased frequency of Porsolt posture (p=0.06), but no difference in latency to enter the Porsolt posture (data not shown).

These results demonstrate that previous testing in paradigms of anxiety-like behaviour significantly increased immobility in the forced swim test. Control rats which had undergone earlier tests of anxiety-like behaviour displayed increased immobility time in the forced swim test compared to naïve counterparts. Similar results were obtained in a group of uncannulated rats, where ‘pre-tested’ rats demonstrated increased immobility compared to the ‘experimentally naïve’ group. The uncannulated ‘pre-tested’ group also displayed a broad variation in immobility (range 14 to 179s; 40% ‘resilient’: 60% ‘depressive’), similar to the cannulated ‘pre-tested’ control rats, whereas all rats in the uncannulated ‘experimentally naïve’ group exhibited ‘resilient’ behaviour (immobility range 5 to 89s).

Analogue 15 treatment produced a significant decrease in immobility in the repeat forced swim test in rats that had undergone previous tests of anxiety-like behaviour, but not in experimentally naïve rats. If an overall score of immobility of <100 s is termed ‘resilient’ and 100 s ‘depressive’, rats in the control group exhibited a broad range of individual variation in immobility, with a 50:50 distribution of resilient/depressive rats (i.e. period of immobility range 19 to 256 s; 45% ‘resilient’ and 55% ‘depressive), whereas all analogue 15-treated rats exhibited ‘resilient’ behaviour (immobility range 4.5 to 85 s).

1. A biologically active relaxin polypeptide comprising a relaxin A chain and a B chain derived from a relaxin superfamily member, wherein the A chain comprises no intra-chain disulphide bonds.

2. The polypeptide of claim 1 wherein the A chain and/or the B chain of the polypeptide is derived from relaxin-3.

3. (canceled)

4. (canceled)

5. The polypeptide of claim 1 wherein the relaxin A chain comprises one or more amino acid substitutions in which one or more cysteine residues responsible for intra-chain disulphide bond formation are replaced by different amino acids.

6. The polypeptide of claim 5 wherein the A chain is derived from human relaxin-3 and one or both of the cysteine residues located at positions 10 and 15, or corresponding positions, of the native relaxin-3 A chain are replaced by alanine residues.

7. The polypeptide of claim 1 wherein the A chain comprises or consists of the amino acid sequence as set forth in SEQ ID NO.3, or a variant or derivative thereof.

8. The polypeptide of claim 1 wherein the polypeptide comprises an A chain comprising or consisting of the amino acid sequence set forth in SEQ ID NO:3, or a variant or derivative thereof and a B chain comprising or consisting of the amino acid sequence set forth in SEQ ID NO:2, or a variant or derivative thereof, and wherein the polypeptide is an agonist of the RXFP3 receptor.

9. The polypeptide of claim 1 wherein the A chain is truncated by up to about 10 amino acids at the N-terminus compared to the corresponding native relaxin A chain sequence.

10. The polypeptide of claim 9 wherein the A chain is derived from relaxin-3 and comprises or consists of the amino acid sequence as set forth in SEQ ID NO.4, or a variant or derivative thereof.

11. The polypeptide of claim 10 wherein the polypeptide comprises an A chain comprising or consisting of the amino acid sequence set forth in SEQ ID NO:4, or a variant or derivative thereof and a B chain comprising or consisting of the amino acid sequence set forth in SEQ ID NO:2, or a variant or derivative thereof, and wherein the polypeptide is an agonist of the RXFP3 receptor.

12. The polypeptide of claim 1 wherein the B chain is truncated by one or more residues at the N-terminus compared to the corresponding native relaxin B chain sequence.

13. The polypeptide of claim 12 wherein the B chain is derived from relaxin-3 and is truncated by up to about 5 residues at the N-terminus, by up to about 7 residues at the N-terminus or up to about 9 residues at the N-terminus.

14. The polypeptide of claim 13 wherein the polypeptide comprises an A chain comprising or consisting of the amino acid sequence set forth in SEQ ID NO:4, or a variant or derivative thereof and a B chain comprising or consisting of the amino acid sequence set forth in SEQ ID NO:6, 7 or 8, or a variant or derivative thereof, and wherein the polypeptide is an agonist of the RXFP3 receptor.

15. The polypeptide of claim 1 wherein: (i) the relaxin-3 derived A chain is truncated by up to about 10 amino acids at the N-terminus compared to the native relaxin-3 A chain sequence; (ii) the relaxin-3 derived B chain is truncated by about 5 amino acids at the C-terminus compared to the native relaxin-3 B chain sequence; and optionally (iii) a basic amino acid such as arginine is incorporated at the C-terminus of the relaxin-3 derived B chain.

16. (canceled)

17. The polypeptide of claim 15 wherein the C-terminal 5 amino acids from the native sequence of the relaxin-3 B chain are replaced by a terminal arginine residue.

18. The polypeptide of claim 17 wherein the B chain comprises or consists of the amino acid sequence set forth in SEQ ID NO:5, or a variant or derivative thereof.

19. The polypeptide of claim 18 comprising an A chain comprising or consisting of the amino acid sequence set forth in SEQ ID NO:4, or a variant or derivative thereof and a B chain comprising or consisting of the amino acid sequence set forth in SEQ ID NO:5, or a variant or derivative thereof, and wherein the polypeptide is an antagonist of the RXFP3 receptor.

20. The polypeptide of claim 1 wherein the polypeptide selectively binds to, or is specific for, the RXFP3 receptor.

21. (canceled)

22. The polypeptide of claim 1, wherein the A chain is derived from relaxin 3, wherein the A chain is truncated by up to 10 amino acids at the N-terminus when compared to the corresponding native relaxin-3 A chain sequence, and wherein the polypeptide is selective for the RXFP3 receptor.

23. (canceled)

24. (canceled)

25. (canceled)

26. The polypeptide of claim 22 wherein the polypeptide is an agonist of the RXFP3 receptor.

27. The polypeptide of claim 1, wherein the B chain comprises a truncation of one or more amino acids from the C-terminus compared to the corresponding native sequence.

28. The polypeptide of claim 27 wherein the A chain is derived from relaxin-3 and is truncated by up to 10 amino acids at the N-terminus when compared to the corresponding native relaxin-3 A chain sequence.

29. (canceled)

30. (canceled)

31. The polypeptide of claim 27 wherein the B chain is truncated by five amino acid residues at the C-terminus.

32. The polypeptide of claim 31 wherein the B chain is derived from relaxin-3 and the terminal five amino acid residues of the B chain are deleted and replaced by a basic amino acid residues such as an arginine residue.

33. (canceled)

34. The polypeptide of claim 27 wherein the polypeptide is an antagonist of the RXFP3 receptor.

35. A polynucleotide encoding a polypeptide according to claim 1.

36. A pharmaceutical composition comprising a polypeptide according to claim 1 or a polynucleotide according to claim 35 optionally together with one or more pharmaceutically acceptable carriers, excipients or diluents.

37. A method for treating or preventing a disease or condition, the method comprising administering to a subject in need thereof a polypeptide according to claim 1 or a polynucleotide according to claim 35 or a pharmaceutical composition according to claim 36.

38. The method of claim 37 wherein the disease or condition is selected from the group consisting of stress, an anxiety disorder, an anxiety-related behavior, a mood disorder, a depressive-related behavior and a depressive symptom of a neurodegenerative disease.

39. The method of claim 38 wherein the anxiety disorder is selected from the group consisting of: global anxiety, chronic anxiety, acute stress disorder, agoraphobia with a history of panic disorder, agoraphobia without a history of panic disorder, generalized anxiety disorder (GAD), obsessive-compulsive disorder (OCD), panic disorder with agoraphobia, panic disorder without agoraphobia, a phobia and posttraumatic stress disorder (PTSD).

40. The method of claim 38 wherein the mood disorder is selected from the group consisting of: bipolar disorder, unipolar disorder, cyclothymic disorder, dysthymic disorder, depression, and major depressive disorder.

41. (canceled)

42. (canceled)
